(19) United States
(12) Patent Application Publication
(10) Pub. No.: US 2023/0110351 A1
(43) Pub. Date: Apr. 13, 2023

(54) METHODS FOR TREATING PARKINSON'S DISEASE WITH SEPIAPTERIN

(71) Applicant: PTC Therapeutics MP, Inc., South Plainfield, NJ (US)

(72) Inventor: Neil SMITH, South Plainfield, NJ (US)

(21) Appl. No.: 17/794,357

(22) PCT Filed: Jan. 22, 2021
(86) PCT No.: PCT/US2021/014745

§ 371 Date: Jul. 21, 2022

Related U.S. Application Data
(60) Provisional application No. 62/965,442, filed on Jan. 24, 2020.

(51) Int. Cl. A61K 31/519; A61K 9/14; A61K 31/197; A61K 9/00; A61P 25/16

(52) U.S. Cl. CPC A61K 31/519; A61K 9/14; A61K 31/197; A61K 9/0053; A61P 25/16

(57) ABSTRACT

The present invention features methods for treating Parkinson's disease using sepiapterin, or a pharmaceutically acceptable salt thereof.

### BACKGROUND OF THE INVENTION

Parkinson's disease is a neurodegenerative disorder that primarily affects dopamine-producing neurons in the substantia nigra and causes a progressive loss of muscle control, leading to trembling of the limbs and head while at rest, stiffness, slowness, and impaired balance. As symptoms worsen, it may become difficult to walk, talk, and complete simple tasks. The disease is accompanied or even preceded by non-motor symptoms that include autonomic dysfunction, gastrointestinal disturbance, neuropsychiatric symptoms affecting mood and cognition, as well as sensory and sleep disturbances.

The symptoms of Parkinson's disease are associated with the greatly reduced activity and number of dopaminergic neurons of the substantia nigra in the midbrain. Lack of dopamine and loss of dopaminergic neuron projections to the striatum lead to activity alterations in the basal ganglia that regulate movement. Dopamine depletion in other non-striatal dopamine pathways is thought to explain some of the neuropsychiatric pathology associated with the disease.

A current treatment for Parkinson's disease is symptomatic and is based on replenishing the dopamine deficiency through the administration of the dopamine metabolic precursor L-3,4-dihydroxyphenylalanine known as levodopa. Levodopa temporarily restores dopamine levels and alleviates dopamine dependent motor and some non-motor symptoms. Despite treatment with levodopa or other dopamine agonists, as the disease progresses, many subjects experience times when levodopa no longer mitigates symptoms and symptoms return; this is known as OFF time when a subject experiences OFF symptoms.

Thus, there remains a need for methods of treatment for both the motor and non-motor symptoms associated with Parkinson's disease, outside the historically used dopamine agonists and levodopa, which minimize the amount of OFF time experienced by a subject.

### SUMMARY OF THE INVENTION

The invention features methods of treating Parkinson's disease in subjects in need thereof by administering sepiapterin, or a pharmaceutically acceptable salt thereof. The inventors have discovered that sepiapterin is effective at increasing tetrahydrobiopterin (BH4), homovanillic acid, 5-hydroxyindoleacetic acid, serotonin, and/or dopamine concentrations (e.g., in the plasma or in the cerebral spinal fluid (CSF)). In an aspect, the invention features a method of treating Parkinson's disease in a subject in need thereof by administering to the subject an effective amount of sepiapterin, or a pharmaceutically acceptable salt thereof, with food (e.g., once per day or twice per day). In an aspect, the invention features a method of treating Parkinson's disease in a subject in need thereof by administering to the subject an effective amount of sepiapterin, or a pharmaceutically acceptable salt thereof, without food (e.g., once per day or twice per day).

In an aspect, the invention features a method of treating Parkinson's disease in a subject in need thereof, by administering to the subject an effective amount of sepiapterin, or a pharmaceutically acceptable salt thereof, more than once per day, e.g., twice per day.

In an aspect, the invention features a method of treating Parkinson's disease in a subject in need thereof, by administering to the subject an effective amount of sepiapterin, or a pharmaceutically acceptable salt thereof, of about 50 mg/kg to about 100 mg/kg (e.g., about 50 mg/kg to about 90 mg/kg, or about 60 mg/kg, about 70 mg/kg, about 80 mg/kg, about 90 mg/kg, or about 100 mg/kg) of sepiapterin or a pharmaceutically acceptable salt per day.

In embodiments of any of the methods described herein, the effective amount of sepiapterin, or a pharmaceutically acceptable salt thereof, is 60 mg/kg per day. In embodiments of any of the methods described herein, the effective amount of sepiapterin, or a pharmaceutically acceptable salt thereof, is 2.5 mg/kg to 100 mg/kg per dose (e.g., about 20 mg/kg to about 80 mg/kg, or about 5 mg/kg, about 10 mg/kg, about 20 mg/kg, about 30 mg/kg, about 40 mg/kg, about 50 mg/kg, about 60 mg/kg, about 70 mg/kg, about 80 mg/kg, or about 90 mg/kg). In embodiments of any of the methods described herein, the effective amount of sepiapterin, or a pharmaceutically acceptable salt thereof, is administered in two equal doses (e.g., two doses at different times of day). In embodiments of any of the methods described herein, the effective amount of sepiapterin, or a pharmaceutically acceptable salt thereof, is administered in two 30 mg/kg doses (e.g., one 30 mg/kg dose in the morning and one 30 mg/kg dose in the evening). In embodiments of any of the methods described herein, the effective amount of sepiapterin, or a pharmaceutically acceptable salt thereof, is 40 mg/kg per day and is administered in two 20 mg/kg doses (e.g., one 20 mg/kg dose in the morning and one 20 mg/kg dose in the evening). In embodiments of any of the methods described herein, the effective amount of sepiapterin, or a pharmaceutically acceptable salt thereof, is 20 mg/kg per day and is administered in two 10 mg/kg doses (e.g., one 10 mg/kg dose in the morning and one 10 mg/kg dose in the evening). In embodiments of any of the methods described herein, the effective amount of sepiapterin, or a pharmaceutically acceptable salt thereof, is administered in two unequal doses (e.g., one 40 mg/kg dose in the morning and one 20 mg/kg dose in the evening). In embodiments of any of the methods described herein, the effective amount of sepiapterin, or a pharmaceutically acceptable salt thereof, is 40 mg/kg per day and is administered in two unequal doses (e.g., one 30 mg/kg dose in the morning and one 10 mg/kg dose in the evening). In embodiments of any of the methods described herein, the effective amount of sepiapterin, or a pharmaceutically acceptable salt thereof, is 20 mg/kg per day and is administered in two unequal doses (e.g., one 15 mg/kg dose in the morning and one 5 mg/kg dose in the evening). In embodiments of any of the methods described herein, the effective amount of sepiapterin, or a pharmaceutically acceptable salt thereof, is administered with a morning meal and with an evening meal (e.g., one dose is administered with the morning meal, and one dose is administered with the evening meal). In embodiments of any of the methods described herein, the effective amount of sepiapterin, or a pharmaceutically acceptable salt thereof, is administered without food twice a day.

In an aspect, the invention features a method of treating Parkinson's disease in a subject in need thereof, by administering to the subject an effective amount of sepiapterin, or a pharmaceutically acceptable salt thereof, in combination with at least one other agent that can be used to treat Parkinson's disease (e.g., levodopa, AADC inhibitors, AADC gene therapy, direct dopamine agonists, MAO-B inhibitors, COMT inhibitors, DAT inhibitors, NMDA antagonists, anticholinergic agents, and acetylcholinesterase inhibitors).

In embodiments of any of the methods described herein, administering sepiapterin, or a pharmaceutically acceptable salt thereof, produces a BH4 concentration of at least 50 ng/mL (e.g., at least 60 ng/mL, at least 100 ng/mL, at least 200 ng/mL, at least 400 ng/mL, at least 600 ng/mL, at least 1000 ng/mL, or at least 2000 ng/mL or from 50 ng/mL to 100 ng/mL, from 60 ng/mL to 400 ng/mL, from 200 ng/mL to 600 ng/mL, from 400 ng/mL to 1000 ng/mL, or from 600 ng/mL to 1500 ng/mL) in the plasma of the subject within 10 hours of administration.

In embodiments of any of the methods described herein, the level of homovanillic acid and/or 5-hydroxyindoleacetic acid in the CSF of the subject is increased (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) compared to the level prior to administration. In embodiments of any of the methods described herein, the effective amount of sepiapterin, or a pharmaceutically acceptable salt thereof, results in an increase in the level of homovanillic acid and/or 5-hydroxyindoleacetic acid in the CSF of the subject of at least 100% (at least 120%, at least 150%, at least 200%, or at least 400%) compared to the level prior to administration. In embodiments of any of the methods described herein, the level of serotonin and/or dopamine is increased (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) in in the CSF of the subject compared to the level prior to administration. In embodiments of any of the methods described herein, the level of serotonin and/or dopamine in the CSF of the subject is increased at least 100% (e.g., at least 120%, at least 150%, at least 200%, or at least 400%) compared to the level prior to administration.

In embodiments of any of the methods herein, the effective amount of sepiapterin, or a pharmaceutically acceptable salt thereof, is a dose that is at least 20% lower (e.g., at least 30% lower, at least 50% lower, at least 70% lower, or at least 90% lower) than the dose sufficient to produce a maximum BH4 plasma concentration (Cₘₐₓ) of at least 50 ng/mL (e.g., at least 60 ng/mL, at least 100 ng/mL, at least 200 ng/mL, at least 400 ng/mL, at least 600 ng/mL, at least 1000 ng/mL, or at least 2000 ng/mL or from 50 ng/mL to 100 ng/mL, from 60 ng/mL to 400 ng/mL, from 200 ng/mL to 600 ng/mL, from 400 ng/mL to 1000 ng/mL, or from 600 ng/mL to 1500 ng/mL) in the plasma of the subject within 10 hours of administration of sepiapterin, or a pharmaceutically acceptable salt thereof, without food.

In embodiments of any of the methods described herein, the method includes administering to the subject an effective amount of sepiapterin, or a pharmaceutically acceptable salt thereof, with food once per day. In embodiments of any of the methods described herein, the method includes administering to the subject an effective amount of sepiapterin, or a pharmaceutically acceptable salt thereof, with food more than once per day, e.g., twice per day. In embodiments of any of the methods described herein, the method includes administering to the subject an effective amount of sepiapterin, or a pharmaceutically acceptable salt thereof, without food once per day. In embodiments of any of the methods described herein, the method includes administering to the subject an effective amount of sepiapterin, or a pharmaceutically acceptable salt thereof, without food more than once per day, e.g., twice per day.

In embodiments of any of the methods described herein, administration of sepiapterin, or a pharmaceutically acceptable salt thereof, to the subject occurs less than 30 minutes prior to consuming food or after consuming food. In embodiments of any of the methods herein, the administration of sepiapterin, or a pharmaceutically acceptable salt thereof, to the subject is substantially at the same time as consuming food. In embodiments of any of the methods described herein, administration to the subject occurs immediately after the consumption of food up to 3 hours, e.g., up to 2 hours, after consuming food (e.g., 10 minutes after, 30 minutes after, 1 hour after, or 2 hours after consuming food). In embodiments of any of the methods described herein, the food is a high protein food. In embodiments of any of the methods described herein, the food is a high fat food (e.g., at least 25, 30, 40, or 50% of the calories are from fat). In embodiments of any of the methods described herein, the food is a high protein and a high fat food. In embodiments of any of the methods herein, the food is high calorie food (e.g., the food includes at least 100 calories, at least 200 calories, at least 300 calories, at least 400 calories, at least 500 calories, e.g., 500-1500 or 800-1000 calories). In embodiments of any of the methods described herein, the food is a meal, e.g., breakfast, lunch, or dinner.

In embodiments of any of the methods described herein, the administration with food (e.g., occurring less than 30 minutes prior to consuming food or after consuming food, e.g., immediately prior to consuming food or up to three hours, e.g., up to two, after consuming food) results in an increase (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) in the Cₘₐₓ of BH4 compared to administration without food (e.g., occurring more than two hours, e.g., more than 3 hours, after consuming food until 30 minutes prior to consuming further food).

In embodiments of any of the methods described herein, the administration with food (e.g., occurring less than 30 minutes prior to consuming food or after consuming food, e.g., immediately prior to the consumption of food up to three hours, e.g., up to two hours, after consumption), results in an increase (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) in the extent of production and resulting plasma exposure (AUC₀₋ₗₐₛₜ) of BH4 compared to administration without food (e.g., occurring more than two hours, e.g., more than three hours, after consuming food until 30 minutes prior to consuming further food).

In embodiments of any of the methods described herein, the administration with food (e.g., occurring less than 30 minutes prior to consuming food or after consuming food, e.g., immediately prior to the consumption of food up to three hours, e.g., up to two hours, after consumption) results in an increase (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) in the Cₘₐₓ of homovanillic acid and/or 5-hydroxyindoleacetic acid compared to administration without food (e.g., occurring more than two hours, e.g., more than three hours, after consuming food until 30 minutes prior to consuming further food).

In embodiments of any of the methods described herein, the administration with food (e.g., occurring less than 30 minutes prior to consuming food or after consuming food, e.g., immediately prior to the consumption of food up to three hours, e.g. up to two hours, after consumption), results in an increase (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) in the extent of production and resulting plasma exposure (AUC₀₋ₗₐₛₜ) of homovanillic acid and/or 5-hydroxyindoleacetic acid compared to administration without food (e.g., occurring more than two hours, e.g., more than three hours, after consuming food until 30 minutes prior to consuming further food).

In embodiments of any of the methods described herein, the administration with food (e.g., occurring less than 30 minutes prior to consuming food or after consuming food, e.g., immediately prior to the consumption of food up to three hours after consumption, e.g., up to two hours after consumption) results in an increase (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) in the Cₘₐₓ of serotonin and/or dopamine compared to administration without food (e.g., occurring more than two hours, e.g., more than three hours, after consuming food until 30 minutes prior to consuming further food).

In embodiments of any of the methods described herein, the administration with food (e.g., occurring less than 30 minutes prior to consuming food or after consuming food, e.g., immediately prior to the consumption of food up to three hours after consumption, e.g., up to two hours after consumption), results in an increase (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) in the extent of production and resulting plasma exposure (AUC₀₋ₗₐₛₜ) of serotonin and/or dopamine compared to administration without food (e.g., e.g., occurring more than two hours, e.g., more than three, after consuming food until 30 minutes prior to consuming further food).

In embodiments of any of the methods described herein, the administration occurs more than 30 minutes prior or at least 2 hours after consuming food (e.g., at least 3 hours, at least 4 hours after consuming food, at least 8 hours after consuming food, or at least 12 hours after consuming food). In some embodiments of any of the methods described herein, the sepiapterin, or pharmaceutically acceptable salt thereof, is administered without a high protein food. In some embodiments of any of the methods described herein, the sepiapterin, or pharmaceutically acceptable salt thereof, is administered without a high fat food (e.g., at least 25, 30, 40, or 50% of the calories are from fat). In some embodiments of any of the methods described herein, the sepiapterin, or pharmaceutically acceptable salt thereof, is administered without a high protein and high fat food. In some embodiments of any of the methods described herein, the sepiapterin, or pharmaceutically acceptable salt thereof, is administered without a high calorie food (e.g., the food includes at least 100 calories, e.g., at least 200 calories, at least 300 calories, at least 400 calories, at least 500 calories, e.g., 500-1500 or 800-1000 calories). In some embodiments of any of the methods described herein, the sepiapterin, or pharmaceutically acceptable salt thereof, is administered without food being a meal, e.g., breakfast, lunch, or dinner.

In some embodiments of any of the methods described herein, the administration without food (e.g., occurring more than about 30 minutes prior to or at least 2 hours, e.g., at least 3 hours, after consuming food) results in an increase (e.g., at least 10% (at least 20%, at least 40%, at least 60%, at least 80%, at least 100%, or at least 120%) in the maximum plasma, CSF, and/or brain concentration (Cₘₐₓ) of sepiapterin compared to administration with food (e.g., occurring less than 30 minutes prior to 3 hours, e.g., to 2 hours, after consuming food).

In some embodiments of any of the methods described herein, the administration without food (e.g., occurring more than 30 minutes prior or at least 2 hours, e.g., at least 3 hours, after consuming food) results in an increase (e.g., at least 10% (at least 20%, at least 40%, at least 60%, at least 80%, at least 100%, or at least 120%) in the extent of absorption (AUC₀₋ₗₐₛₜ) of sepiapterin compared to administration with food (e.g., the administration to the subject occurs less than 30 minutes prior to less than 3 hours, e.g., less than 2 hours, after consuming food).

In embodiments of any of the methods described herein, the sepiapterin, or pharmaceutically acceptable salt thereof, is formulated as an oral powder for suspension. In embodiments of the methods described herein, the sepiapterin, or a pharmaceutically acceptable salt thereof, is administered as a suspension in a flavored suspending vehicle (e.g., Medisca Oral Mix). In embodiments of any of the methods described herein, the sepiapterin, or a pharmaceutically acceptable salt thereof, is administered as a suspension in water. In embodiments of the methods described herein, the sepiapterin, or a pharmaceutically acceptable salt thereof, is administered as a suspension in juice (e.g., apple, grape, or orange juice). In embodiments of any of the methods described herein, the sepiapterin, or pharmaceutically acceptable salt thereof, is formulated as an oral capsule, tablet, caplet, or similar dosage form.

In embodiments of any of the methods described herein, the method further includes administering an effective amount of levodopa to the subject. In embodiments of any of the methods described herein, the method further includes administering an effective amount of an AADC inhibitor, e.g., carbidopa, to the subject. In embodiments of any of the methods described herein, the method further includes administering an effective amount of a COMT inhibitor, a dopamine agonist, a MAO-B inhibitor, a NMDA antagonist, an AADC inhibitor, a DAT inhibitor, an anticholinergic agent, and/or an acetylcholinesterase inhibitor.

In embodiments of any of the methods described herein, administration of the effective amount of sepiapterin, or a pharmaceutically acceptable salt thereof, is for at least 1 week (e.g., at least 2 weeks, at least 1 month, at least 2 months, at least 6 months, or at least 1 year). In embodiments of any of the methods described herein, administration results in an improvement in the MDS-UPDRS of the subject, such that the overall score is lower (e.g., at least 10% lower, at least 20% lower, at least 30% lower, at least 40% lower, at least 50% lower, at least 60% lower, at least 70% lower, at least 80% lower, or at least 90% lower) for a subject after having been administered sepiapterin, or a pharmaceutically acceptable salt thereof, compared to before having been administered sepiapterin, or a pharmaceutically acceptable salt thereof. In embodiments of any of the methods described herein, administration results in a decrease (e.g., at least a 10% decrease, at least a 20% decrease, at least a 30% decrease, at least a 40% decrease, at least a 50% decrease, at least a 60% decrease, at least a 70% decrease, at least a 80% decrease, or at least a 90% decrease) in the Total Daily OFF Time in the subject, such that the total number of hours per day wherein the treatment administered to the subject does not adequately manage the symptoms associated with Parkinson's is less after a subject is administered sepiapterin, or a pharmaceutically acceptable salt thereof, compared to before a subject is administered sepiapterin, or a pharmaceutically acceptable salt thereof. In embodiments of any of the methods described herein, administration results in an improvement in the Hamilton Depression Rating Scale in the subject, such that the score measured for a subject after being administered sepiapterin, or a pharmaceutically acceptable salt thereof, is lower (e.g., at least 10% lower, at least 20% lower, at least 30% lower, at least 40% lower, at least 50% lower, at least 60% lower, at least 70% lower, at least 80% lower, or at least 90% lower) on an integer scale of 0 to 52, wherein 0 is considered no depression and 52 is considered severe depression, than before being administered sepiapterin, or a pharmaceutically acceptable salt thereof. In embodiments of any methods described herein, administrations result in an improvement in the Parkinson's Disease Sleep Scale in the subject, such that the score measured for a subject after being administered sepiapterin, or a pharmaceutically acceptable salt thereof, is higher (e.g., at least 10% higher, at least 20% higher, at least 30% higher, at least 40% higher, at least 50% higher, at least 60% higher, at least 70% higher, at least 80% higher, at least 90% higher, or at least 100% higher) on an integer scale of 0 to 10 for 15 points of assessment, wherein 0 is awful sleep and 10 is excellent sleep, compared to before being administered sepiapterin, or a pharmaceutically acceptable salt thereof.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) where ranges are provided, endpoints are included.

It is to be understood that the description of compounds, compositions, formulations, and methods of treatment described herein include “comprising”, “consisting of”, and “consisting essentially of” embodiments. In some embodiments, for all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as ‘consisting essentially of’ a component, the composition may additionally contain any amount of pharmaceutically acceptable carriers, vehicles, or diluents and other such components which do not substantially affect the condition being treated.

As used herein, the term “about” represents a value that is in the range of ±10% of the value that follows the term “about.” Reference to “about” a value or parameter herein includes (and describes) variations that are directed to that value or parameter per se. For example, description referring to “about X” includes description of “X”.

As used herein, the term “administration” refers to the administration of a composition (e.g., a compound or a preparation that includes a compound as described herein) to a subject or system.

Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

The term “Cₘₐₓ,” as used herein, refers to an observed maximum plasma concentration produced in a subject administered sepiapterin, or a pharmaceutically acceptable salt thereof.

An “effective amount” of a compound may vary according to factors such as the disease state, age, sex, and weight of the individual, and the ability of the compound to elicit the desired response. A “therapeutically effective amount” encompasses an amount in which any toxic or detrimental effects of the compound are outweighed by the therapeutically beneficial effects. A therapeutically effective amount also encompasses an amount sufficient to confer benefit, e.g., clinical benefit.

The term “food,” as used herein, refers to solid food with sufficient bulk and fat content that it is not rapidly dissolved and absorbed in the stomach. For example, a meal, such as breakfast, lunch, or dinner. The term “with food,” as used herein refers to administration of a composition between about 30 minutes prior to about three hours after, e.g., between about 30 minutes prior to about two hours after, eating, e.g., a meal. The terms “without food” or “fasted” refer to the condition of not having consumed solid food for at least about two, e.g., at least about three hours, after until about 30 minutes prior to consuming further solid food.

By “level” is meant a level of a compound, as compared to a reference. The reference can be any useful reference, as defined herein, e.g., the level in the subject prior to administration, i.e., a baseline level. By a “decreased level” or an “increased level” of a compound is meant a decrease or increase in the level as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a compound may be expressed in mass/vol (e.g., g/dL, mg/mL, μg/mL, ng/mL), concentration/vol (e.g., nanomole per liter, micromole per liter, micromole per milliliter), or percentage relative to other compounds, e.g., total amino acids, in a sample.

The terms “OFF,” “OFF time,” and “OFF symptoms” as used herein, refer to when medication used to treat the symptoms associated with Parkinson's disease is no longer working optimally, resulting in times when the symptoms, e.g., motor fluctuations, return.

The term “pharmaceutical composition,” as used herein, represents a composition containing a compound described herein formulated with a pharmaceutically acceptable excipient. A pharmaceutical composition may be one manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gel cap, powder for suspension, suspension, solution, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

As used herein, the term “pharmaceutically acceptable salt” means any salt that within the scope of sound medical judgment is suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The compounds of the invention may have ionizable groups so as to be capable of preparation as pharmaceutically acceptable salts. These salts may be acid addition salts involving inorganic or organic acids, or the salts may, in the case of acidic forms of the compounds of the invention, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. The salts can be prepared in situ during the final isolation and purification of the compounds described herein or separately by reacting a free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. As used herein, the term “substantially free” refers to the qualitative condition of exhibiting total or near-total extent or degree of the absence of a compound or type of compound of interest. One of ordinary skill in the biological arts will understand that biological and chemical phenomena rarely, if ever, can be determined to be zero without doubt, e.g., due to inherent error in any measurement. The term “substantially free” is therefore used herein to capture the potential lack of completeness inherent in many biological and chemical measurements.

As used herein, the term “subject” or “patient” or “participant” refers to any organism to which a compound or composition in accordance with the invention may be administered, e.g., for experimental, diagnostic, prophylactic, and/or therapeutic purposes. Typical subjects include any animal (e.g., mammals such as mice, rats, rabbits, dogs, non-human primates, and humans). A subject may seek or be in need of treatment, require treatment, be receiving treatment, be receiving treatment in the future, or be a human or animal who is under care by a trained professional for a particular disease or condition. The subject may be currently experiencing symptoms associated with Parkinson's disease or may have experienced them in the past, or may be experiencing one or more symptoms of Parkinson's disease, even though a diagnosis may not have been made.

As used herein, the terms “treat,” “treated,” or “treating” mean both therapeutic treatment and prophylactic or preventive measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the subject; or enhancement or improvement of condition, disorder, or disease. Treatment includes eliciting a clinically significant response without excessive levels of side effects. Treatment also includes prolonging survival as compared to expected survival if not receiving treatment.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the description and from the claims.

### DETAILED DESCRIPTION

The present inventors have discovered that sepiapterin, is surprisingly effective at treating Parkinson's disease. In some embodiments, the sepiapterin is administered with food. In some embodiments, the sepiapterin is administered without food. In particular, the inventors have discovered that sepiapterin can be effective at increasing levels (e.g., plasma levels or CSF levels) of BH4, homovanillic acid, 5-hydroxyindoleacetic acid, serotonin, and/or dopamine. Accordingly, the present invention features methods for treating Parkinson's disease in a subject in need thereof by administering sepiapterin, or a pharmaceutically acceptable salt thereof.

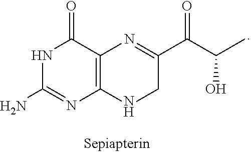 [SMILES: C[C@H](O)C(=O)C1=Nc2c(nc(N)[nH]c2=O)NC1]

Sepiapterin, or a pharmaceutically acceptable salt thereof, can be formulated in a pharmaceutical composition. In some embodiments, a pharmaceutical composition of the invention includes 20-30% sepiapterin, or a salt thereof, by total weight, e.g., 20%, 22%, 25%, 27%, or 30%. In some embodiments, the pharmaceutical compositions include greater than 20% sepiapterin by total weight, e.g., greater than 25%, greater than 30%, greater than 40%, greater than 50%, greater than 60%, greater than 70%, greater than 80%, or greater than 90%. In some embodiments, the pharmaceutical composition includes less than 20% sepiapterin by total weight, e.g., less than 20%, less than 15%, less than 10%, or less than 5%.

In some embodiments, the invention features a pharmaceutical composition including sepiapterin, or a salt thereof, and less than 10% by total weight of an antioxidant, e.g., about 9%, 7%, 5%, 3%, 1%, 0.5%, 0.25%, 0.1%, or no antioxidant. The antioxidant may be ascorbic acid. In some embodiments, the ratio of sepiapterin, or a pharmaceutically acceptable salt thereof, to the antioxidant is 1:1, or greater than 1:1, e.g., 2:1, 5:1, 7:1, or 10:1 by weight. The pharmaceutical composition may include 20-30% sepiapterin, or a pharmaceutically acceptable salt thereof, by total weight, e.g., about 20%, 22%, 25%, 27%, or 30%. The pharmaceutical composition can further include a dispersant, e.g., croscarmellose sodium. The pharmaceutical composition may include 0.1-1.5% dispersant by total weight, e.g., 0.1%, 0.5%, 1%, or 1.5%. In some embodiments, the pharmaceutical composition includes at least one anti-caking agent, e.g., colloidal silicon dioxide or microcrystalline cellulose. The pharmaceutical composition may include 65-75% anti-caking agent by total weight, e.g., about 65%, 67%, 70%, 73%, or 75%. In some embodiments, the pharmaceutical composition includes both colloidal silicon dioxide and microcrystalline cellulose. In some embodiments, the pharmaceutical composition includes 60-65% microcrystalline cellulose by total weight and 5-7% colloidal silicon dioxide by total weight. In some embodiments, sepiapterin is formulated as particles less than 140 μm in size, e.g., about 120 μm, 110 μm, 100 μm, 90 μm, 80 μm, 70 μm, 60 μm, 50 μm, 40 μm, 30 μm, 20 μm, 10 μm, or 5 μm. In some embodiments, the pharmaceutical composition includes less than 1% of an impurity by total weight such as lactoylpterin, e.g., the composition includes less than 0.9%, less than 0.8%, less than 0.7%, less than 0.6%, less than 0.5%, less than 0.4%, less than 0.3%, or less than 0.2%.

In some embodiments, the sepiapterin is a salt of sepiapterin. In preferred embodiments, the sepiapterin is a salt of sepiapterin with hydrochloric acid, sulfuric acid, p-toluene sulfonic acid, methane sulfonic acid, benzene sulfonic acid, malonic acid, tartaric acid (e.g., L-tartaric acid), phosphoric acid, gentisic acid, fumaric acid, glycolic acid, acetic acid, or nicotinic acid.

In some embodiments, the sepiapterin, or pharmaceutically acceptable salt thereof, is in crystalline form. The crystalline sepiapterin free base or a crystalline form of a salt of sepiapterin can occur as an anhydrate (e.g., without having any bound water or solvent or hydration or solvation) or as a hydrate, a partial hydrate (e.g., hemihydrate, sesquihydrate), as a dihydrate, or a trihydrate, wherein the crystalline form binds a water of hydration or a solvent molecule associated with the crystalline form of sepiapterin or salt thereof. In an embodiment, crystalline sepiapterin occurs as a monohydrate or as a hemihydrate. Exemplary salts, co-crystals, and crystalline forms of sepiapterin are described in International Publication Nos: WO 2018/102314, WO 2018/102315, WO 2019/232120, and WO 2019/046849, the crystalline forms, salts, and co-crystals of which are incorporated herein by reference in their entirety.

In some embodiments, sepiapterin free base is present in a crystalline form. In some embodiments, the crystalline form of sepiapterin free base is crystalline Form F of sepiapterin free base and is characterized by an X-ray powder diffraction pattern obtained by irradiation with Cu Kα X-rays having peaks expressed as 2θ at 9.7°±0.5, e.g., 9.7°±0.2, 10.2°±0.5, e.g., 10.2°±0.2, and 11.3°±0.5, e.g., 11.3°±0.2. In other embodiments, the crystalline Form F of sepiapterin free base is characterized by an X-ray powder diffraction pattern obtained by irradiation with Cu Kα X-rays having peaks expressed as 2θ at 9.7°±0.5, e.g., 9.7°±0.2, 10.2°±0.5, e.g., 10.2°±0.2, 11.3°±0.5, e.g., 11.3°±0.2, 14.0°±0.5, e.g., 14.0°±0.2, 14.6°±0.5, e.g., 14.6°±0.2, 19.9°±0.5, e.g., 19.9°±0.2, 22.2°±0.5, e.g., 22.2°±0.2, 25.3°±0.5, e.g., 25.3°±0.2, and 32.4°±0.5, e.g., 32.4°±0.2. In an essentially pure form of this crystalline form, peaks can be observed at angles of refraction 2θ as set forth in Table 1. Alternatively or in addition, this crystalline form is characterized by a DSC curve showing two endotherms at 71.6° C. and 233.4° C.

TABLE 1

| Position [2θ°] (±0.5, e.g., ±0.2) | Relative Intensity |
|---|---|
| 9.7 | 98.27 |
| 10.2 | 100.00 |
| 11.3 | 22.47 |
| 14.0 | 5.01 |
| 14.6 | 12.36 |
| 19.9 | 5.63 |
| 21.1 | 3.72 |
| 22.2 | 5.37 |
| 22.7 | 4.04 |
| 24.5 | 2.99 |
| 25.3 | 17.65 |
| 27.2 | 3.10 |
| 32.4 | 5.29 |
| 36.7 | 2.72 |

In some embodiments, the crystalline form of sepiapterin free base is crystalline Form B of sepiapterin free base and has peaks at diffraction angle 2θ (°) of 8.4°±0.5, e.g., 8.4°±0.2, 16.9°±0.5, e.g., 16.9°±0.2, and 25.4°±0.5, e.g., 25.4°±0.2 as measured by X-ray diffractometry by irradiation with Cu Kα X-rays or calculated from X-ray diffractometry. In some embodiments, the crystalline Form B of sepiapterin free base has peaks at diffraction angle 2θ (°) of 8.4°±0.5, e.g., 8.4°±0.2, 14.9°±0.5, e.g., 14.9°±0.2, 16.9°±0.5, e.g., 16.9°±0.2, 25.4°±0.5, e.g., 25.4°±0.2, and 34.1°±0.5, e.g., 34.10°±0.2 as measured by X-ray diffractometry by irradiation with Cu Kα X-rays or calculated from X-ray diffractometry. In an essentially pure material of this crystalline form, peaks can be observed at angles of refraction 2θ as set forth in Table 2. Alternatively, or in addition, this crystalline form is characterized by a DSC curve showing a melting event at 195.2° C.

TABLE 2

| Position [2θ°] (±0.5, e.g., ±0.2) | Relative Intensity |
|---|---|
| 8.4 | 100.00 |
| 14.9 | 2.34 |
| 16.9 | 10.70 |
| 25.4 | 84.90 |
| 34.1 | 3.00 |

In some embodiments, the crystalline form of sepiapterin free base is crystalline Form C of sepiapterin free base and has peaks at diffraction angle 2θ (°) of 5.7°±0.5, e.g., 5.7°±0.2, 7.8°±0.5, e.g., 7.8°±0.2, and 25.4°±0.5, e.g., 25.4°±0.2 as measured by X-ray diffractometry by irradiation with Cu Kα X-rays or calculated from X-ray diffractometry. In some embodiments, the crystalline Form C of sepiapterin free base has peaks at diffraction angle 2θ (°) of 5.7°±0.5, e.g., 5.7°±0.2, 7.8°±0.5, e.g., 7.8°±0.2, 9.1°±0.5, e.g., 9.1°±0.2, 11.5°±0.5, e.g., 11.5°±0.2, 15.3°±0.5, e.g., 15.3°±0.2, 16.0°±0.5, e.g., 16.0°±0.2, 20.1°±0.5, e.g., 20.1°±0.2, 25.4°±0.5, e.g., 25.4°±0.2, and 26.6°±0.5, e.g., 26.6°±0.2, as measured by X-ray diffractometry by irradiation with Cu Kα X-rays or calculated from X-ray diffractometry. In an essentially pure material of this crystalline form, peaks can be observed at angles of refraction 2θ as set forth in Table 3. Alternatively or in addition, this crystalline form is characterized by a DSC curve showing five endothermal peaks at 58.3° C., 101.8° C., 129.8° C., 156.5° C., and 168.3° C.

TABLE 3

| Position [2θ°] (±0.5, e.g., ±0.2) | Relative Intensity |
|---|---|
| 5.7 | 48.91 |
| 7.8 | 100.00 |
| 9.1 | 59.49 |
| 10.4 | 8.72 |
| 11.5 | 24.53 |
| 12.9 | 8.50 |
| 14.8 | 9.24 |
| 15.3 | 12.53 |
| 16.0 | 14.09 |
| 17.2 | 7.22 |
| 18.2 | 4.25 |
| 19.2 | 5.78 |
| 20.1 | 14.54 |
| 21.5 | 6.47 |
| 22.9 | 6.85 |
| 23.7 | 4.80 |
| 25.4 | 65.68 |
| 26.6 | 14.53 |
| 27.4 | 8.39 |
| 31.5 | 3.74 |
| 34.2 | 4.36 |

In some embodiments, the crystalline form of sepiapterin free base is crystalline Form D of sepiapterin free base and has peaks at diffraction angle 2θ (°) of 8.9°±0.5, e.g., 8.9°±0.2, 10.3°±0.5, e.g., 10.3°±0.2, and 26.0°±0.5, e.g., 26.0°±0.2 as measured by X-ray diffractometry by irradiation with Cu Kα X-rays or calculated from X-ray diffractometry. In some embodiments, the crystalline Form D of sepiapterin free base has peaks at diffraction angle 2θ (°) of 8.9°±0.5, e.g., 8.9°±0.2, 10.3°±0.5, e.g., 10.3°±0.2, 10.9°±0.5, e.g., 10.9°±0.2, 178°±0.5, e.g., 17.8°±0.5, 24.9°±0.5, e.g., 24.9°±0.2, 26.0°±0.5, e.g., 26.0°±0.2, 26.7°±0.5, e.g., 26.7°±0.2, 26.8°±0.5, e.g., 26.8°±0.2, and 28.3°±0.5, e.g., 28.3°±0.2, as measured by X-ray diffractometry by irradiation with Cu Kα X-rays or calculated from X-ray diffractometry. In an essentially pure material of this crystalline form, peaks can be observed at angles of refraction 2θ as set forth in Table 4. Alternatively or in addition, this crystalline form is characterized by a DSC curve showing three endotherms at 42.7° C., 66.3° C., and 232.9° C.

TABLE 4

| Position [2θ°] (±0.5, e.g., ±0.2) | Relative Intensity |
|---|---|
| 8.9 | 100.00 |
| 10.3 | 49.92 |
| 10.9 | 19.96 |
| 11.6 | 2.15 |
| 13.6 | 2.99 |
| 14.2 | 3.45 |
| 14.8 | 2.35 |
| 15.4 | 2.59 |
| 16.4 | 1.55 |
| 17.2 | 2.33 |
| 17.8 | 6.24 |
| 19.6 | 2.62 |
| 20.1 | 2.28 |
| 20.5 | 3.09 |
| 20.8 | 2.27 |
| 21.3 | 3.60 |
| 22.3 | 4.79 |
| 23.7 | 4.31 |
| 24.9 | 5.19 |
| 26.0 | 41.94 |
| 26.7 | 8.58 |
| 26.8 | 9.17 |
| 27.4 | 3.98 |
| 28.3 | 4.75 |
| 28.7 | 6.60 |
| 29.8 | 3.03 |
| 31.8 | 2.72 |
| 33.0 | 2.03 |
| 35.5 | 1.57 |
| 37.1 | 1.09 |

In some embodiments, the crystalline form of sepiapterin free base is crystalline Form A of sepiapterin free base and has peaks at diffraction angle 2θ (°) of 4.7°±0.5, e.g., 4.7°±0.2, 7.4°±0.5, e.g., 7.4°±0.2, and 26.2°±0.5, e.g., 26.2°±0.2 as measured by X-ray diffractometry by irradiation with Cu Kα X-rays or calculated from X-ray diffractometry. In some embodiments, the crystalline form A of sepiapterin free base has at least one peak at diffraction angle 2θ (°) of 4.7°±0.5, e.g., 4.7°±0.2, 7.4°±0.5, e.g., 7.4°±0.2, 9.5°±0.5, e.g., 9.5°±0.2, 11.3°±0.5, e.g., 11.3°±0.2, 15.6°±0.5, e.g., 15.6°±0.2, 16.4°±0.5, e.g., 16.4°±0.2, 26.2°±0.5, e.g., 26.2°±0.2, and 27.2°±0.5, e.g., 27.2°±0.2 as measured by X-ray diffractometry by irradiation with Cu Kα X-rays or calculated from X-ray diffractometry. In an essentially pure material of this crystalline form, peaks can be observed at angles of refraction 2θ as set forth in Table 5. Alternatively, or in addition, this crystalline form is characterized by a DSC curve showing endothermal peaks at 82.8° C. and 179.8° C.

TABLE 5

| Position [2θ°] (±0.5, e.g., ±0.2) | Relative Intensity |
|---|---|
| 4.7 | 47.76 |
| 7.4 | 100.00 |
| 9.5 | 33.54 |
| 11.3 | 19.31 |
| 12.4 | 8.49 |
| 13.4 | 3.60 |
| 14.2 | 8.24 |
| 15.6 | 15.08 |
| 16.4 | 11.97 |
| 17.6 | 8.35 |
| 18.4 | 5.03 |
| 19.8 | 9.18 |
| 21.5 | 5.44 |
| 24.4 | 5.56 |
| 26.2 | 35.37 |
| 27.2 | 19.11 |
| 28.9 | 5.93 |

In some embodiments, the crystalline form of sepiapterin free base is crystalline Form E of sepiapterin free base and has at peaks at diffraction angle 2θ (°) of 6.0°±0.5, e.g., 6.0°±0.2, 10.6°±0.5, e.g., 10.6°±0.2, 12.1°±0.5, e.g., 12.1°±0.2, 15.9°±0.5, e.g., 15.9°±0.2, 20.9°±0.5, e.g., 20.9°±0.2, and 24.6°±0.5, e.g., 24.6°±0.2 as measured by X-ray diffractometry by irradiation with Cu Kα X-rays or calculated from X-ray diffractometry. In some embodiments, the crystalline Form E of sepiapterin free base has peaks at diffraction angle 2θ (°) of 6.0°±0.5, e.g., 6.0°±0.2, 10.6°±0.5, e.g., 10.6°±0.2, 12.1°±0.5, e.g., 12.1°±0.2, 15.9°±0.5, e.g., 15.9°±0.2, 18.10°±0.5, e.g., 18.1°±0.2, 20.9°±0.5, e.g., 20.9°±0.2, 22.1°±0.5, e.g., 22.1°±0.2, 24.6°±0.5, e.g., 24.6°±0.2, 26.1°±0.5, e.g., 26.1°±0.2, 28.1°±0.5, e.g., 28.1°±0.2, 28.9°±0.5, e.g., 28.9°±0.2, 32.1°±0.5, e.g., 32.1°±0.2, and 37.0°±0.5, e.g., 37.0°±0.2 as measured by X-ray diffractometry by irradiation with Cu Kα X-rays or calculated from X-ray diffractometry. In an essentially pure form of this crystalline form, peaks can be observed at angles of refraction 2θ as set forth in Table 6. Alternatively or in addition, this crystalline form is characterized by a DSC curve showing two endothermal peaks at 112.9° C. and 195.8° C.

TABLE 6

| Position [2θ°] (±0.5, e.g., ±0.2) | Relative Intensity |
|---|---|
| 6.0 | 100.00 |
| 10.6 | 20.78 |
| 12.1 | 31.95 |
| 15.9 | 12.83 |
| 18.1 | 3.39 |
| 20.9 | 11.63 |
| 22.1 | 2.79 |
| 24.6 | 8.28 |
| 26.1 | 0.88 |
| 28.1 | 7.33 |
| 28.9 | 3.77 |
| 32.1 | 3.57 |
| 37.0 | 1.03 |

In some embodiments, the crystalline form of sepiapterin free base is crystalline Form G of sepiapterin free base and has peaks at diffraction angle 2θ (°) of 10.0°±0.5, e.g., 10.0°±0.2, 10.6°±0.5, e.g., 10.6°±0.2, and 25.7°±0.5, e.g., 25.7°±0.2 as measured by X-ray diffractometry by irradiation with Cu Kα X-rays or calculated from X-ray diffractometry. In some embodiments, the crystalline Form G of sepiapterin free base has peaks at diffraction angle 2θ (°) of 10.0°±0.5, e.g., 10.0°±0.2, 10.6°±0.5, e.g., 10.6°±0.2, 11.2°±0.5, e.g., 11.2°±0.2, 15.3°±0.5, e.g., 15.3°±0.2, 15.9°±0.5, e.g., 15.9°±0.2, 22.8°±0.5, e.g., 22.8°±0.2, 24.4°±0.5, e.g., 24.4°±0.2, 25.0°±0.5, e.g., 25.0°±0.2, 25.7°±0.5, e.g., 25.7°±0.2, and 26.6°±0.5, e.g., 26.6°±0.2 as measured by X-ray diffractometry by irradiation with Cu Kα X-rays or calculated from X-ray diffractometry. In an essentially pure material of this crystalline form, peaks can be observed at angles of refraction 2θ as set forth in Table 7.

TABLE 7

| Position [2θ°] (±0.5, e.g., ±0.2) | Relative Intensity |
|---|---|
| 5.3 | 8.30 |
| 6.9 | 4.54 |
| 10.0 | 100.00 |
| 10.6 | 69.64 |
| 11.2 | 6.59 |
| 13.5 | 7.52 |
| 15.3 | 26.59 |
| 15.9 | 26.43 |
| 16.0 | 23.41 |
| 16.9 | 4.28 |
| 18.6 | 13.02 |
| 19.3 | 11.90 |
| 20.1 | 7.22 |
| 20.8 | 11.01 |
| 22.8 | 16.77 |
| 23.5 | 19.60 |
| 24.4 | 41.45 |
| 25.0 | 23.99 |
| 25.7 | 65.40 |
| 26.6 | 39.64 |
| 27.6 | 13.04 |
| 28.7 | 6.55 |
| 30.8 | 14.76 |
| 32.2 | 9.63 |
| 33.7 | 5.16 |
| 37.5 | 5.80 |

In some embodiments, the crystalline form of the hydrochloride salt of sepiapterin has peaks at diffraction angle 2θ (°) of 7.8°±0.5, e.g., 7.8°±0.2, 12.9°±0.5, e.g., 12.9°±0.2, and 26.2°±0.5, e.g., 26.2°±0.2, as measured by X-ray diffractometry by irradiation with Cu Kα X-rays or calculated from X-ray diffractometry. In some embodiments, the most intense peak in the X-ray diffraction diagram of the crystalline form of the hydrochloride salt of sepiapterin is observed at an angle of refraction 2θ of 7.8°±0.5, e.g., 7.8°±0.2. In an essentially pure material of this crystalline hydrochloride salt of sepiapterin, peaks can be observed at angles of refraction 2θ as set forth in Table 8. Alternatively or in addition, the crystalline hydrochloride salt of sepiapterin is characterized by a DSC curve showing an endotherm at 225.9° C.

TABLE 8

| Position [2θ°] (±0.5, e.g., ±0.2) | Relative Intensity |
|---|---|
| 7.8 | 100.00 |
| 8.9 | 6.89 |
| 12.9 | 58.56 |
| 15.6 | 8.52 |
| 17.9 | 25.23 |
| 19.2 | 5.48 |
| 21.1 | 10.97 |
| 23.6 | 25.15 |
| 25.2 | 22.66 |
| 26.2 | 45.91 |
| 27.6 | 32.94 |
| 30.3 | 10.50 |
| 31.7 | 7.83 |
| 34.2 | 8.87 |
| 36.7 | 3.67 |

In some embodiments, the crystalline Form 1 methanesulfonate salt of sepiapterin has peaks at the diffraction angle 2θ (°) of 7.8°±0.5, e.g., 7.8°±0.2, 23.5°±0.5, e.g. 23.5°±0.2, and 29.7°±0.5, e.g., 29.0°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of 23.5°±0.5, e.g., 23.5°±0.2. In an essentially pure material of the crystalline Form 1 methanesulfonate salt of sepiapterin, peaks can be observed at angles of refraction 2θ as set forth in Table 9. Alternatively or in addition, the crystalline form 1 methanesulfonate salt of sepiapterin is characterized by a DSC curve showing two endotherms at 186.0° C. and 229.1° C.

TABLE 9

| Position [2θ°] (±0.5, e.g., ±0.2) | Relative Intensity |
|---|---|
| 7.9 | 21.77 |
| 11.7 | 8.20 |
| 13.7 | 8.52 |
| 15.7 | 4.79 |
| 16.6 | 5.34 |
| 18.0 | 5.66 |
| 19.8 | 2.10 |
| 20.3 | 5.36 |
| 20.9 | 2.43 |
| 22.3 | 4.25 |
| 22.7 | 2.15 |
| 23.5 | 100.00 |
| 24.7 | 3.69 |
| 25.6 | 2.70 |
| 26.8 | 1.79 |
| 27.2 | 1.68 |
| 28.3 | 2.75 |
| 29.0 | 57.60 |
| 29.8 | 5.18 |
| 30.5 | 1.37 |
| 32.2 | 4.66 |
| 33.0 | 1.64 |
| 36.5 | 1.29 |

In some embodiments, the crystalline Form 2 methanesulfonate salt of sepiapterin has peaks at the diffraction angle 2θ (°) of 7.9°±0.5, e.g., 7.9°±0.2, 23.4°±0.5, e.g., 23.4°±0.2 and 28.9°±0.5, e.g., 28.9°±0.2. as measured by X-ray diffractometry by irradiation with Cu Kα X-rays or calculated from X-ray diffractometry. In some embodiments, the most intense peak in the X-ray diffraction diagram is observed at an angle of refraction 2θ of 7.9°±0.5, e.g., 7.9°±0.2. In an essentially pure material of the crystalline Form 2 methanesulfonate salt of sepiapterin, peaks can be observed at angles of refraction 2θ as set forth in Table 10. Alternatively or in addition, the crystalline form 2 methanesulfonate salt of sepiapterin is characterized by a DSC curve showing three endotherms at 75.5° C., 182.6° C., and 234.9° C.

TABLE 10

| Position [2θ°] (±0.5, e.g., ±0.2) | Relative Intensity |
|---|---|
| 7.9 | 100.00 |
| 11.0 | 21.32 |
| 12.1 | 22.02 |
| 13.5 | 79.87 |
| 15.7 | 11.87 |
| 17.8 | 9.81 |
| 19.7 | 10.93 |
| 21.3 | 26.79 |
| 23.4 | 96.13 |
| 24.1 | 24.88 |
| 24.3 | 22.10 |
| 25.5 | 9.45 |
| 26.0 | 11.27 |
| 27.6 | 7.63 |
| 28.9 | 95.64 |
| 31.2 | 4.39 |
| 36.1 | 6.65 |

In some embodiments, the crystalline Form 3 methanesulfonate salt of sepiapterin has peaks at the diffraction angle 2θ (°) of 21.7°±0.5, e.g., 21.7°±0.2, 26.0°±0.5, e.g., 26.0°±0.2, and 28.9°±0.5, e.g., 28.9°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of 26.0°±0.5, e.g., 26.0°±0.2. In an essentially pure material of the crystalline Form 3 methanesulfonate salt of sepiapterin, peaks can be observed at angles of refraction 2θ as set forth in Table 11. Alternatively or in addition, the crystalline form 3 methanesulfonate salt of sepiapterin is characterized by a DSC curve showing two endotherms at 195.1° C. and 240.1° C.

TABLE 11

| Position [2θ°] (±0.5, e.g., ±0.2) | Relative Intensity |
|---|---|
| 8.2 | 47.29 |
| 10.8 | 56.14 |
| 12.6 | 16.34 |
| 13.2 | 15.90 |
| 14.0 | 24.39 |
| 15.0 | 12.03 |
| 15.9 | 16.20 |
| 18.2 | 22.97 |
| 20.1 | 25.53 |
| 20.5 | 14.97 |
| 21.3 | 22.70 |
| 21.7 | 71.48 |
| 22.2 | 11.40 |
| 23.6 | 46.37 |
| 24.8 | 44.00 |
| 25.5 | 9.08 |
| 26.1 | 100.00 |
| 27.3 | 3.52 |
| 28.9 | 68.42 |
| 31.2 | 4.49 |
| 32.1 | 6.48 |
| 34.8 | 5.95 |
| 35.6 | 1.67 |
| 39.1 | 2.91 |

In some embodiments, the crystalline nicotinate salt of sepiapterin has peaks at the diffraction angle 2θ (°) of 9.5°±0.5, e.g., 9.5°±0.2, 9.9°±0.5, e.g., 9.9°±0.2, and 24.5°±0.5, e.g., 24.5°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of 24.5°±0.5, e.g., 24.5°±0.2. In an essentially pure material of the crystalline nicotinate salt of sepiapterin, peaks can be observed at angles of refraction 2θ as set forth in Table 12. Alternatively or in addition, the crystalline nicotinate salt of sepiapterin is characterized by a DSC curve showing an endotherm at 221.9° C.

TABLE 12

| Position [2θ°] (±0.5, e.g., ±0.2) | Relative Intensity |
|---|---|
| 9.5 | 10.29 |
| 9.9 | 53.95 |
| 11.5 | 9.31 |
| 12.0 | 11.76 |
| 14.7 | 14.20 |
| 15.9 | 17.61 |
| 17.5 | 7.53 |
| 19.0 | 5.37 |
| 20.8 | 5.88 |
| 21.3 | 6.12 |
| 21.7 | 7.20 |
| 23.2 | 34.05 |
| 24.5 | 100.00 |
| 25.2 | 12.90 |
| 28.0 | 8.51 |
| 31.1 | 5.39 |
| 32.3 | 4.52 |
| 33.4 | 8.02 |
| 35.1 | 5.05 |

In some embodiments, the crystalline p-toluenesulfonate salt of sepiapterin has peaks at the diffraction angle 2θ (°) of 6.5°±0.5, e.g., 6.5°±0.2, 15.1°±0.5, e.g., 15.1°±0.2, and 23.4°±0.5, e.g., 23.4°±0.2 as measured by X-ray diffractometry by irradiation with Cu Kα X-rays or calculated from X-ray diffractometry. In some embodiments, the most intense peak in the X-ray diffraction diagram is observed at an angle of refraction 2 of 6.5°±0.5, e.g., 6.5°±0.2. In an essentially pure material of the p-toluenesulfonate salt of sepiapterin, peaks can be observed at angles of refraction 2θ as set forth in Table 13. Alternatively or in addition, the crystalline p-toluenesulfonate salt of sepiapterin is characterized by a DSC curve showing three endotherms at 77.2° C., 202.4° C. and 260.2° C.

TABLE 13

| Position [2θ°] (±0.5, e.g., ±0.2) | Relative Intensity |
|---|---|
| 6.5 | 100.00 |
| 12.9 | 1.79 |
| 14.3 | 1.39 |
| 15.1 | 15.36 |
| 16.2 | 5.33 |
| 18.4 | 8.96 |
| 19.6 | 3.06 |
| 20.2 | 4.86 |
| 21.8 | 2.23 |
| 22.5 | 2.95 |
| 23.1 | 7.99 |
| 23.4 | 9.14 |
| 24.5 | 1.81 |
| 26.0 | 2.48 |
| 27.0 | 4.49 |
| 27.3 | 3.93 |
| 28.1 | 5.31 |
| 28.4 | 5.59 |
| 28.8 | 2.05 |
| 30.6 | 2.24 |
| 31.0 | 1.98 |
| 32.6 | 1.82 |

In some embodiments, the crystalline benzenesulfonate salt of sepiapterin has peaks at the diffraction angle 2θ (°) of 6.5°±0.5, e.g., 6.5°±0.2, 14.8°±0.5, e.g., 14.8°±0.2, and 19.6°±0.5, e.g., 19.6°±0.2 as measured by X-ray diffractometry by irradiation with Cu Kα X-rays or calculated from X-ray diffractometry. In some embodiments, the most intense peak in the X-ray diffraction diagram is observed at an angle of refraction 2 of 6.5°±0.5, e.g., 6.5°±0.2. In an essentially pure material of the benzenesulfonate salt of sepiapterin, peaks can be observed at angles of refraction 2θ as set forth in Table 14. Alternatively or in addition, the crystalline benzenesulfonate salt of sepiapterin is characterized by a DSC curve showing two endotherms at 202.3° C. and 265.5° C.

TABLE 14

| Position [2θ°] (±0.5, e.g., ±0.2) | Relative Intensity |
|---|---|
| 4.9 | 5.90 |
| 6.5 | 100.00 |
| 14.8 | 16.73 |
| 17.8 | 4.23 |
| 19.6 | 7.98 |
| 21.5 | 2.49 |
| 23.7 | 3.46 |
| 24.5 | 3.84 |
| 26.1 | 3.29 |

In some embodiments, the crystalline phosphate salt of sepiapterin has peaks at the diffraction angle 2θ (°) of 16.6°±0.5, e.g., 16.6°±0.2, 22.2°±0.5, e.g., 22.2°±0.2, and 25.6°±0.5, e.g., 25.6°±0.2 as measured by X-ray diffractometry by irradiation with Cu Kα X-rays or calculated from X-ray diffractometry. In some embodiments, the most intense peak in the X-ray diffraction diagram is observed at an angle of P refraction 2θ of 25.6°±0.5, e.g., 25.6°±0.2. In an essentially pure material of the crystalline phosphate salt of sepiapterin, peaks can be observed at angles of refraction 2θ as set forth in Table 15. Alternatively or in addition, the crystalline phosphate salt of sepiapterin is characterized by a DSC curve showing three endotherms at 125.9° C., 152.1° C., and 157.6° C.

TABLE 15

| Position [2θ°] (±0.5, e.g., ±0.2) | Relative Intensity |
|---|---|
| 5.5 | 4.41 |
| 8.1 | 1.21 |
| 8.9 | 2.21 |
| 10.3 | 1.79 |
| 10.8 | 5.80 |
| 15.3 | 1.84 |
| 16.6 | 8.35 |
| 17.7 | 1.95 |
| 20.3 | 1.40 |
| 21.2 | 1.61 |
| 22.2 | 9.77 |
| 23.1 | 1.74 |
| 25.6 | 100.00 |
| 30.8 | 6.31 |
| 31.1 | 4.85 |
| 33.5 | 0.73 |
| 36.0 | 1.70 |

In some embodiments, the crystalline malonate salt of sepiapterin has peaks at the diffraction angle 2θ (°) of 6.9°±0.5, e.g., 6.9°±0.2, 22.7°±0.5, e.g., 22.7°±0.2 and 23.8°±0.5, e.g., 23.8°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of 6.9°±0.5, e.g., 6.9°±0.2. In an essentially pure material of the crystalline malonate salt of sepiapterin, peaks can be observed at angles of refraction 2θ as set forth in Table 16. Alternatively or in addition, the crystalline malonate salt of sepiapterin is characterized by a DSC curve showing a melting event at 115.8° C.

TABLE 16

| Position [2θ°] (±0.5, e.g., ±0.2) | Relative Intensity |
|---|---|
| 6.9 | 100.00 |
| 8.4 | 13.11 |
| 10.6 | 7.62 |
| 16.4 | 5.63 |
| 17.8 | 9.73 |
| 19.3 | 8.96 |
| 20.1 | 9.99 |
| 22.2 | 10.50 |
| 22.7 | 20.52 |
| 23.8 | 34.02 |
| 24.5 | 5.82 |
| 25.5 | 24.50 |
| 26.6 | 4.00 |
| 27.3 | 6.96 |
| 29.8 | 5.38 |
| 33.1 | 12.08 |

In some embodiments, the crystalline L-tartrate salt of sepiapterin has peaks at the diffraction angle 2θ (°) of 7.4°±0.5, e.g., 7.4°±0.2, 14.2°±0.5, e.g., 14.2°±0.2, and 21.8°±0.5, e.g., 21.8°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of 7.4°±0.5, e.g., 7.4°±0.2. In an essentially pure material of the crystalline L-tartrate salt of sepiapterin, peaks can be observed at angles of refraction 2θ as set forth in Table 17. Alternatively or in addition, the crystalline L-tartrate salt of sepiapterin is characterized by a DSC curve showing two endotherms at 97.2° C. and 160.6° C.

TABLE 17

| Position [2θ°] (±0.5, e.g., ±0.2) | Relative Intensity |
|---|---|
| 7.4 | 100.00 |
| 10.1 | 47.99 |
| 14.2 | 82.76 |
| 14.7 | 27.06 |
| 19.1 | 21.16 |
| 20.2 | 29.91 |
| 21.8 | 85.30 |
| 22.1 | 53.68 |
| 23.9 | 85.30 |
| 24.9 | 19.26 |
| 25.5 | 28.45 |
| 26.8 | 18.58 |
| 29.7 | 21.59 |
| 31.6 | 10.10 |
| 32.9 | 22.18 |

In some embodiments, the crystalline gentisate salt of sepiapterin has peaks at the diffraction angle 2θ (°) of 7.1°±0.5, e.g., 7.1°±0.2, 8.7°±0.5, e.g., 8.7°±0.2, and 26.7°±0.5, e.g., 26.7°±0.2 as measured by X-ray diffractometry by irradiation with Cu Kα X-rays or calculated from X-ray diffractometry. In some embodiments the most intense peak in the X-ray diffraction diagram is observed at an angle of refraction 2θ of 7.1°±0.5, e.g., 7.10°±0.2. In an essentially pure material of the crystalline gentisate salt of sepiapterin, peaks can be observed at angles of refraction 2θ as set forth in Table 18. Alternatively or in addition, the crystalline gentisate salt of sepiapterin is characterized by a DSC curve showing three endotherms at 70.5° C., 128.2° C., and 184.7° C.

TABLE 18

| Position [2θ°] (±0.5, e.g., ±0.2) | Relative Intensity |
|---|---|
| 5.7 | 17.29 |
| 7.1 | 100.00 |
| 8.7 | 42.69 |
| 10.4 | 3.94 |
| 11.3 | 11.69 |
| 12.1 | 4.13 |
| 14.3 | 21.10 |
| 16.0 | 6.46 |
| 16.4 | 5.94 |
| 17.0 | 5.85 |
| 17.6 | 7.93 |
| 19.1 | 8.27 |
| 20.20 | 3.47 |
| 20.7 | 2.90 |
| 21.5 | 3.37 |
| 23.6 | 2.69 |
| 24.4 | 4.50 |
| 26.7 | 52.20 |
| 27.1 | 35.49 |
| 28.2 | 8.74 |
| 28.9 | 4.31 |
| 29.9 | 2.62 |
| 31.4 | 2.99 |
| 34.4 | 1.28 |

In some embodiments, the crystalline fumarate salt of sepiapterin has peaks at the diffraction angle 2θ (°) of 11.3±0.5, e.g., 11.3°±0.2, 24.0°±0.5, e.g., 24.0°±0.2 and 28.2°±0.5, e.g., 28.2°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of at least 24.0°±0.5, e.g., 24.0°±0.2. In an essentially pure material of the crystalline fumarate salt of sepiapterin, peaks can be observed at angles of refraction 2θ as set forth in Table 19. Alternatively or in addition, the crystalline fumarate salt of sepiapterin is characterized by a DSC curve showing two endotherms at 114.3° C. and 229.7° C.

TABLE 19

| Position [2θ°] (±0.5, e.g., ±0.2) | Relative Intensity |
|---|---|
| 6.1 | 6.43 |
| 7.7 | 5.40 |
| 11.4 | 53.62 |
| 11.9 | 33.37 |
| 14.2 | 8.03 |
| 16.5 | 6.70 |
| 18.3 | 13.86 |
| 19.0 | 6.68 |
| 20.7 | 10.02 |
| 21.3 | 7.02 |
| 22.8 | 24.68 |
| 24.0 | 100.00 |
| 28.3 | 33.26 |
| 32.7 | 6.35 |
| 36.0 | 3.28 |
| 38.5 | 6.02 |

In some embodiments, the crystalline glycolate salt of sepiapterin has peaks at the diffraction angle 2θ (°) of 7.6°±0.5, e.g., 7.6°±0.2, 10.7°±0.5, e.g., 10.7°±0.2, and 24.0°±0.5, e.g., 24.0°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of 7.6°±0.5, e.g., 7.6°±0.2. In an essentially pure material of the crystalline glycolate salt of sepiapterin, peaks can be observed at angles of refraction 2 as set forth in Table 20. Alternatively or in addition, the crystalline glycolate salt of sepiapterin is characterized by a DSC curve showing two endotherms at 133.9° C. and 147.7° C.

TABLE 20

| Position [2θ°] (±0.5, e.g., ±0.2) | Relative Intensity |
|---|---|
| 4.8 | 6.23 |
| 7.6 | 100.00 |
| 10.3 | 68.06 |
| 10.7 | 70.69 |
| 15.3 | 36.51 |
| 18.2 | 24.25 |
| 18.7 | 27.26 |
| 19.9 | 2.66 |
| 21.2 | 17.11 |
| 24.0 | 96.62 |
| 24.4 | 18.44 |
| 28.8 | 47.57 |
| 30.3 | 7.43 |
| 32.5 | 4.42 |
| 33.3 | 7.49 |
| 34.3 | 5.21 |
| 36.3 | 7.37 |

In some embodiments, the crystalline acetate salt has peaks at the diffraction angle 2θ (°) of 6.2°±0.5, e.g., 6.2°±0.2, 12.0°±0.5, e.g. 12.0°±0.2, and 18.1°±0.5, e.g., 18.1°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of at least 6.2°±0.5, e.g., 6.2°±0.2. In an essentially pure material of the crystalline acetate salt of sepiapterin, peaks can be observed at angles of refraction 2θ as set forth in Table 21. Alternatively or in addition, the crystalline acetate salt of sepiapterin is characterized by a DSC curve showing two endotherms at 146.10° C. and 175.4° C.

TABLE 21

| Position [2θ°] (±0.5, e.g., ±0.2) | Relative Intensity |
|---|---|
| 6.2 | 100.00 |
| 10.2 | 23.29 |
| 12.0 | 71.59 |
| 18.1 | 31.27 |
| 21.1 | 20.29 |
| 24.2 | 14.92 |
| 25.2 | 23.03 |
| 27.3 | 13.30 |
| 29.1 | 12.95 |

In some embodiments, the crystalline Form 1 sulfate salt of sepiapterin has peaks at the diffraction angle 2θ (°) of 5.1°±0.5, e.g., 5.1°±0.2, 7.8°±0.5, e.g., 7.8°±0.2, and 23.0°±0.5, e.g., 23.0°±0.2 as measured by X-ray diffractometry by irradiation with Cu Kα X-rays or calculated from X-ray diffractometry. In some embodiments, the most intense peak in the X-ray diffraction diagram is observed at an angle of refraction 2 of 5.1°±0.5, e.g., 5.1°±0.2 In an essentially pure material of the crystalline Form 1 sulfate salt of sepiapterin, peaks can be observed at angles of refraction 2θ as set forth in Table 22. Alternatively or in addition, the crystalline form 1 sulfate salt of sepiapterin is characterized by a DSC curve showing three endotherms at 94.5° C., 158.3° C., and 209.9° C.

TABLE 22

| Position [2θ°] (±0.5, e.g., ±0.2) | Relative Intensity |
|---|---|
| 5.1 | 100.00 |
| 6.8 | 3.33 |
| 7.8 | 43.48 |
| 10.2 | 15.92 |
| 15.7 | 18.13 |
| 17.2 | 8.33 |
| 18.7 | 6.49 |
| 19.8 | 5.19 |
| 21.3 | 5.52 |
| 23.0 | 19.05 |
| 23.5 | 8.29 |
| 24.2 | 5.59 |
| 24.8 | 17.44 |
| 25.7 | 4.97 |
| 26.7 | 10.38 |
| 28.7 | 11.49 |
| 30.4 | 2.88 |
| 31.0 | 3.67 |

In some embodiments, the crystalline Form 2 sulfate salt of sepiapterin has peaks at the diffraction angle 2θ (°) of 7.8°±0.5, e.g., 7.8°±0.2, 8.8°±0.2, e.g., 8.8°±0.2, and 24.1°±0.5, e.g., 24.1°±0.2 as measured by X-ray diffractometry by irradiation with Cu Kα X-rays or calculated from X-ray diffractometry. In some embodiments, the most intense peak in the X-ray diffraction diagram is observed at an angle of refraction 2θ of 8.8°±0.5, e.g., 8.8°±0.2. In an essentially pure material of the crystalline Form 2 sulfate salt of sepiapterin, peaks can be observed at angles of refraction 2θ as set forth in Table 23.

TABLE 23

| Position [2θ°] (±0.5, e.g., ±0.2) | Relative Intensity |
|---|---|
| 5.0 | 4.71 |
| 7.9 | 72.24 |
| 8.8 | 100.00 |
| 14.5 | 19.26 |
| 15.7 | 59.40 |
| 16.1 | 8.69 |
| 17.2 | 14.82 |
| 17.7 | 10.89 |
| 19.3 | 9.92 |
| 20.2 | 9.60 |
| 23.7 | 15.38 |
| 24.2 | 43.88 |
| 25.0 | 11.44 |
| 26.8 | 16.81 |
| 28.7 | 16.07 |
| 29.4 | 13.84 |
| 31.3 | 17.14 |
| 31.7 | 7.26 |
| 35.7 | 5.75 |

The present invention provides a pharmaceutical composition including a pharmaceutically acceptable excipient and an effective amount of sepiapterin, or a pharmaceutically acceptable salt thereof. The pharmaceutically acceptable excipient can be any of those conventionally used and is limited only by chemico-physical considerations, such as solubility and by the route of administration. It will be appreciated by one of skill in the art that, in addition to the following described pharmaceutical compositions, sepiapterin, or a pharmaceutically acceptable salt thereof, can be formulated as inclusion complexes, such as cyclodextrin inclusion complexes, or liposomes.

The pharmaceutically acceptable excipients described herein, for example, vehicles, adjuvants, excipients, or diluents, are well known to those who are skilled in the art and are readily available to the public. It is preferred that the pharmaceutically acceptable excipient be one which is chemically inert to the sepiapterin, or a pharmaceutically acceptable salt thereof, and one which has no detrimental side effects or toxicity under the conditions of use.

Sepiapterin, or a pharmaceutically acceptable salt thereof, can be used in the preparation of liquid formulations, such as in the form of a solution, suspension, or emulsion. Formulations suitable for oral administration can include, but are not limited to, of (a) capsules, sachets, tablets, lozenges, and troches, each containing a predetermined amount of the active ingredient, as solids or granules; (b) powders; (c) liquid solutions, such as an effective amount of the compound dissolved in diluents, such as water, saline, or orange juice; (d) suspensions in an appropriate liquid; and (e) suitable emulsions. Preferred are solid oral dosage forms such as capsule forms, tablet forms, and powder forms. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are known in the art.

Formulations suitable for oral and/or parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical excipient, such as a sterile liquid or mixture of liquids, including water, saline, aqueous dextrose and related sugar solutions, an alcohol, such as ethanol, benzyl alcohol, or hexadecyl alcohol, glycols, such as propylene glycol or polyethylene glycol and other polyethylene alcohols, glycerol ketals, such as 2,2-dimethyl-1,3-dioxolane-4-methanol, ethers, such as poly(ethylene glycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

The present invention features pharmaceutical compositions in an orally tolerable formula that contains a therapeutically effective amount of sepiapterin, or a pharmaceutically acceptable salt thereof, and less than 10% antioxidant. In some embodiments, the pharmaceutical composition is a granular formulation that is dispersed in a pharmaceutically acceptable excipient, for example the composition can be mixed into water and ingested by a subject (e.g., over the course of 5 to 10 minutes). Suitable formulations for use in the present invention are found in Remington's Pharmaceutical Sciences, Mack Publishing Company, Philadelphia, Pa. 22nd ed., 2010. Except insofar as any conventional excipient is incompatible with the active ingredient, its use in the pharmaceutical compositions is contemplated. Moreover, for animal (e.g., human) administration, it will be understood that preparations should meet sterility, pyrogenicity, general safety and purity standards as required by FDA Office of Biological Standards.

Formulations which Increase Gastric and/or Anterior Intestine Residence Time

Gastro-retentive drug delivery is an approach with the drug formulation is designed to remain in the stomach longer, e.g., until drug release is complete.

Bioadhesive dosage forms utilize polymers that are capable of adhering to surfaces and result in a controlled release of the drug. The bioadhesive polymers may be anionic (e.g., carboxymethylcellulose, alginic acid, polyacrylic acid, pectin, carrageenan, polycarbophil, or carbomer); cationic (e.g., chitosan, polylysine, or polybrene); or non-ionic (e.g., polyethylene glycol, polyvinylpyrrolidone, dextran, orhydroxypropylmethylcellulose).

High-density dosage forms are designed to sit in the stomach at a lower level than the pyloric sphincter, and thus avoid emptying. Excipients suitable for high-density dosage forms include iron powder, barium sulphate, zinc oxide, and titanium oxide.

Expandable dosage forms are designed to expand in the stomach to be larger than the pyloric sphincter, and thus avoid emptying. For example, dosage forms including a drug core, a swellable hydrocolloid, and an outer semi-permeable polymer are suitable for expandable dosage forms.

Super-porous hydrogel dosage forms are designed, similarly to expandable dosage forms, to expand in the stomach to be larger than the pyloric sphincter. Super-porous hydrogel dosage forms may include polymers such as croscarmellose sodium.

Floating dosage forms are designed to have a lower density than gastric fluid. Floating dosage forms may include compositions including ion exchange resin, a raft system, an inflatable chamber, an effervescent mixture, a swellable hydrocolloid, or a multi-particulate system.

Antioxidants

Sepiapterin is prone to rapid oxidation when exposed to air. Accordingly, pharmaceutical compositions of the invention may include antioxidants. The antioxidant may minimize the oxidative degradation of sepiapterin. In some embodiments, examples of antioxidants include, but are not limited to, 4-chloro-2,6-di-tert-butylphenol, tocopherol, alpha-tocopherol, alkylated diphenylamines, ascorbic acid, ascorbyl myristate, ascorbyl palmitate, ascorbyl stearate, beta-carotene, butylated hydroxyanisole, butylated hydroxytoluene, citric acid, cysteine, D-alpha-tocopheryl polyethylene glycol 1°±0.0 succinate, deferoxamine methanesulfonate, dodecyl gallate, ethylparaben, folic acid, fumaric acid, gallic acid, glutathione, lecithin, malic acid, methylparaben, monothioglycerol, N-acetyl cysteine, nordihydroguaiaretic acid, octyl gallate, p-phenylenediamine, potassium ascorbate, potassium metabisulfite, potassium sorbate, propionic acid, propyl gallate, retinol, sorbic acid, sodium ascorbate, sodium bisulfite, sodium hydrosulfite, sodium isoascorbate, sodium metabisulfite, sodium sulfite, sodium thiosulfate, tartaric acid, tert-butylhydroquinone, tocopheryl acetate, vitamin A, vitamin B6, vitamin B112, or vitamin E. In some embodiments, the antioxidants include, but are not limited to, ascorbic acid, tocopherol, retinol, ascorbyl palmitate, N-acetyl cysteine, glutathione, ethylenediaminetetraacetic acid, sodium bisulfite, sodium metabisulfite, thiourea, butylatedhydroxytoluene, butylatedhydroxyanisole, and vitamin E. In some embodiments, the pharmaceutical compositions of the invention include ascorbic acid, tocopherol, retinol, ascorbyl palmitate, N-acetyl cysteine, glutathione, butylatedhydroxytoluene, and/or butylatedhydroxyanisole as antioxidant.

In some embodiments, the pharmaceutical composition includes less than 10% antioxidant by weight, e.g., less than 9%, less than 8%, less than 7%, less than 6%, less than 5%, less than 4%, less than 3%, less than 2%, less than 1%, or substantially free of antioxidant. In some embodiments, the pharmaceutical composition includes 2-9% antioxidant by total weight, e.g., 2-4%, 3-5%, 4-6%, 5-7%, 6-8%, or 7-9%. In some embodiments, the pharmaceutical composition comprises 5-100% of the USP maximum daily dose of the antioxidant, e.g., in some embodiments, the pharmaceutical composition comprises about 5%, 10%, 15%, 20%, 30%, 40%, 50%, 60%, 70%, 80%, 90%, or 100% of the USP maximum daily dose of the antioxidant. In some embodiments, the ratio of sepiapterin, or a pharmaceutically acceptable salt thereof, to antioxidant is at least 1:1, e.g., 2:1, 3:1, 4:1, 5:1, 6:1, 7:1, 8:1, 9:1, or 10:1 by weight.

Dispersants

In some embodiments, the pharmaceutical compositions of the invention include at least one dispersant. The dispersant may cause particles in the formulation to separate, e.g., release their medicinal substances on contact with moisture. Examples of dispersants include, but are not limited to, crosslinked polyvinylpyrrolidone, carboxymethylcellulose (e.g., croscarmellose salt, e.g., croscarmellose sodium), starch (e.g., sodium starch glycolate), or alginic acid. In some embodiments, the dispersant in the pharmaceutical composition is a carboxymethylcellulose such as a pharmaceutically acceptable salt of croscarmellose. In some embodiments, the pharmaceutical composition may include 0.1-1.5% dispersant by total weight, e.g., about 0.1%, 0.5%, 1%, or 1.5%. In some embodiments, the pharmaceutical composition includes less than 1.5% dispersant, e.g., less than 1%, less than 0.5%, or less than 0.1% by total weight.

Anti-Caking Agents

In some embodiments, the pharmaceutical compositions of the invention include at least one anti-caking agent. In some embodiments, the pharmaceutical compositions include at least two anti-caking agents. Exemplary anti-caking agents include colloidal silicon dioxide, microcrystalline cellulose, tricalcium phosphate, microcrystalline cellulose, magnesium stearate, sodium bicarbonate, sodium ferrocyanide, potassium ferrocyanide, calcium ferrocyanide, calcium phosphate, sodium silicate, colloidal silicon dioxide, calcium silicate, magnesium trisilicate, talcum powder, sodium aluminosilicate, potassium aluminum silicate, calcium aluminosilicate, bentonite, aluminum silicate, stearic acid, and polydimethylsiloxane. In some embodiments, the at least one anti-caking agent is colloidal silicon dioxide or microcrystalline cellulose. In some embodiments, the pharmaceutical composition may include 65-75% anti-caking agent by total weight, e.g., about 65%, 67%, 70%, 73%, or 75%. In some embodiments, the pharmaceutical composition includes both colloidal silicon dioxide and microcrystalline cellulose. In some embodiments, the pharmaceutical composition includes 60-65% microcrystalline cellulose by total weight and 5-7% colloidal silicon dioxide by total weight.

Dosing Vehicle

In some embodiments, the pharmaceutical compositions of the invention are combined with a dosing vehicle prior to administration, e.g., a dosing vehicle with a viscosity of approximately 50-1750 centipoise (cP). One type of suspending agent that can be used is a combination of glycerin and sucrose in water (e.g., Medisca oral mix with 2.5% glycerin and 27% sucrose in water). An appropriate quantity of composition can be added to the dosing vehicle mixture and agitated to suspend the composition just prior to administration.

Other suspending agents may also be used as a dosing vehicle. Exemplary suspending agents include water, agar, alginic acid, sodium carboxymethyl cellulose, carrageenan, dextrin, gelatin, guar gum, hydroxyethyl cellulose, hydroxypropyl cellulose, hypromellose, methyl cellulose, polyethylene glycol, povidone, tragacanth, xanthan gum, or other suspending agents known in the art.

Solid Dosage Form of Oral Administration

Formulations for oral use may include particles containing the active ingredient(s) in a mixture with non-toxic pharmaceutically acceptable excipients, and such formulations are known to the skilled artisan (e.g., U.S. Pat. Nos. 5,817,307, 5,824,300, 5,830,456, 5,846,526, 5,882,640, 5,910,304, 6,036,949, 6,036,949, 6,372,218, hereby incorporated by reference). Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, anti-adhesives (e.g., magnesium stearate, zinc stearate, stearic acid, silicas, hydrogenated vegetable oils, or talc), and anti-caking agents (e.g., colloidal silicon dioxide, microcrystalline cellulose, tricalcium phosphate, microcrystalline cellulose, magnesium stearate, sodium bicarbonate, sodium ferrocyanide, potassium ferrocyanide, calcium ferrocyanide, calcium phosphate, sodium silicate, colloidal silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane). Other pharmaceutically acceptable excipients can be colorants, flavoring agents, plasticizers, humectants, and buffering agents. In some embodiments, excipients (e.g., flavoring agents) are packaged with the composition. In some embodiments, excipients (e.g., flavorings) are packaged separately from the composition (e.g., are combined with the composition prior to administration).

The solid compositions of the invention may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology, eds. J. Swarbrick and J. C. Boylan, 2006, Marcel Dekker, New York, which is incorporated herein by reference.

Powders and granulates may be prepared using the ingredients mentioned above in a conventional manner using, e.g., a mixer, a fluid bed apparatus, melt congeal apparatus, rotor granulator, extrusion/spheronizer, or spray drying equipment.

Dosage Sepiapterin, or pharmaceutically acceptable salt thereof, can be used in any suitable dose.

Suitable doses and dosage regimens can be determined by conventional range finding techniques. For convenience, the total daily dosage may be divided and administered in portions during the day if desired. In proper doses and with suitable administration of certain compounds, the present invention provides for a wide range of responses. A total daily dosage of sepiapterin, or a pharmaceutically acceptable salt thereof, may range from about 50 mg/kg to about 100 mg/kg body weight of the subject per day, e.g., 60 mg/kg/day. Typically, the dosages range from about 2.5 mg/kg to about 100 mg/kg body weight of the subject being treated per dose, e.g., 30 mg/kg/dose. For example, in embodiments, a dose of sepiapterin, or a pharmaceutically acceptable salt thereof, may be administered from about 10 mg/kg to about 100 mg/kg, from about 20 mg/kg to about 90 mg/kg, from about 30 mg/kg to about 80 mg/kg, from about 40 mg/kg to about 70 mg/kg, from about 40 mg/kg to about 100 mg/kg, from about 50 mg/kg to about 90 mg/kg, from about 30 mg/kg to about 70 mg/kg, from about 2.5 mg/kg to about 80 mg/kg, from about 2.5 mg/kg to about 80 mg/kg, or from about 2.5 mg/kg to about 70 mg/kg, of subject body weight per dose, one or more times a day (e.g., twice a day, three times a day, or four times a day), to obtain the desired therapeutic effect.

In some embodiments, the sepiapterin, or pharmaceutically acceptable salt thereof, can be formulated into unit solid oral dosage forms such as particles. In these embodiments, each unit solid oral dosage form, e.g., sachet, can include any suitable amount of the sepiapterin, or pharmaceutically acceptable salt thereof. For example, each solid oral dosage form can include about 2.5 mg, about 5 mg, about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 750 mg, about 1 g, about 1.25 g, or about 1.5 g.

Compositions for Combination Therapy

A compound of the invention can be used alone or in combination with other agents that can be used to treat neurodegenerative disorders, such as Parkinson's disease. A compound of the invention can be admixed with an additional active agent and administered to a subject in a single composition (e.g., a tablet or capsule), or a compound of the invention can be administered to a subject separately from an additional active agent. A compound of the invention and an additional active agent can be sequentially administered to a subject as part of a dosing regimen described herein. For instance, a compound of the invention may be admixed or formulated for co-administration with levodopa (L-dihydroxyphenylalanine), L-aromatic amino acid decarboxylase (AADC) inhibitors, AADC gene therapy, catechol-O-methyltransferase (COMT) inhibitors, dopamine transporter (DAT) inhibitors, monoamine oxidase (MAO-B) inhibitors, N-methyl-D-aspartate (NMDA) receptor antagonists, acetylcholinesterase inhibitors, anticholinergic agents, and/or dopamine agonists. Exemplary AADC inhibitors that may be included in a pharmaceutical composition of the invention include carbidopa ((2S)-3-(3,4-dihydroxyphenyl)-2-hydrazinyl-2-methylpropanoic acid) and benserazide (2-amino-3-hydroxy-N′-[(2,3,4-trihydroxyphenyl)methyl]propanehydrazide). Exemplary COMT inhibitors that may be included in a pharmaceutical composition of the invention include entacapone ((E)-2-cyano-3-(3,4-dihydroxy-5-nitrophenyl)-N,N-diethylprop-2-enamide), tolcapone ((3,4-dihydroxy-5-nitrophenyl)-(4-methylphenyl)methanone), and/or nitecapone (3-[(3,4-dihydroxy-5-nitrophenyl)methylidene]pentane-2,4-dione). Exemplary DAT inhibitors that may be included in the invention include bicifadine, 3α-bis-(4-flurophenyl) methoxytropane, bupropion, 3-CPMT, duloxetine, indatraline, reserpine, rimcazole, and/or tetrabenazine. Exemplary MAO-B inhibitors that may be included in the invention include rasagiline and selegiline. Exemplary acetylcholinesterase inhibitors that may be included in the invention include donepezil, galantamine, and memantine. Exemplary NMDA antagonists that may be included in the invention include pethidine, levorphanol, methadone, dextropropoxyphene, tramadol, ketobemidone, memantine, and/or amantadine. Exemplary dopamine agonists that may be included in the invention include bromocriptine, mirtazapine, ropinirole, pramipexole, rotigotine, and apomorphine. Exemplary anticholinergic agents that may be included in the invention include benztropine, clomipramine, fluphenazine, loxapine, pimozide, olanzapine, and/or trihexyphenidyl. In some embodiments, subjects continue their current treatments for Parkinson's disease (e.g., levodopa, AADC inhibitors, dopamine agonists, MAO-B inhibitors, COMT inhibitors, anticholinergics, acetylcholinesterase inhibitors, DAT inhibitors, and/or NMDA receptor antagonists). In some embodiments, subjects may not be permitted to take any drugs known to inhibit folate synthesis (e.g., methotrexate, pemetrexed, and/or trimetrexate).

In combination treatments, the dosages of one or more of the therapeutic compounds may be reduced from standard dosages when administered alone. For example, doses may be determined empirically from drug combinations and permutations or may be deduced by isobolographic analysis (e.g., Black et al., Neurology 65:S3-S6, 2°±0.5). In this case, dosages of the compounds when combined may provide a therapeutic effect.

### Methods of Treatment

Sepiapterin, or a pharmaceutically acceptable salt thereof, may serve as a useful therapeutic for treating Parkinson's disease. Thus, the various forms of sepiapterin, or pharmaceutically acceptable salts thereof, in accordance with the present invention can be administered to a subject in an effective amount to obtain treatment of the disease.

The actual dosage amount of a composition of the present invention administered to a subject can be determined by physical and physiological factors such as body weight, severity of condition, the type of disease being treated, previous or concurrent therapeutic interventions, idiopathy of the subject, and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In some embodiments, subjects receive about 2.5 mg/kg to 100 mg/kg per dose (e.g., about 20 mg/kg to about 80 mg/kg, or about 20 mg/kg, about 30 mg/kg, about 40 mg/kg, about 50 mg/kg, about 60 mg/kg, about 70 mg/kg, about 80 mg/kg, about 90 mg/kg). Subjects may receive the pharmaceutical composition including sepiapterin, or a pharmaceutically acceptable salt thereof, once daily, twice daily, three times daily, or four times daily during treatment. Sepiapterin, or a pharmaceutically acceptable salt thereof, may be administered in two equal doses (e.g., two doses at different times of day), e.g., two 60 mg/kg doses (e.g., one 60 mg/kg dose in the morning and one 60 mg/kg dose in the evening), two 40 mg/kg doses (e.g., one 40 mg/kg dose in the morning and one 40 mg/kg dose in the evening), two 30 mg/kg doses (e.g., one 30 mg/kg dose in the morning and one 30 mg/kg dose in the evening), two 20 mg/kg doses (e.g., one 20 mg/kg dose in the morning and one 20 mg/kg dose in the evening), or two 10 mg/kg doses (e.g., one 10 mg/kg dose in the morning and one 10 mg/kg dose in the evening).

In some embodiments, subjects who are taking BH4 discontinue administration of BH4 (i.e., BH4 washout, e.g., prior to or concomitant with initiation of sepiapterin treatment). Blood samples for BH4 concentrations may be obtained during the BH4 washout period at 7, 5, 3, and 1 day before the treatment with the pharmaceutical composition of the invention.

Subjects treated by the methods herein may have a BH4 concentration of at least 50 ng/mL (e.g., at least 60 ng/mL, at least 100 ng/mL, at least 200 ng/mL, at least 400 ng/mL, at least 600 ng/mL, at least 1000 ng/mL, or at least 2000 ng/mL or from 50 ng/mL to 100 ng/mL from 60 ng/mL to 400 ng/mL, from 200 ng/mL to 600 ng/mL, from 400 ng/mL to 1000 ng/mL, or from 600 ng/mL to 1500 ng/mL) in the plasma of the subject within 10 hours of administration.

Administration of sepiapterin, or a pharmaceutically acceptable salt thereof, to the subject by any of the methods described herein may result in an increase in concentration of homovanillic acid and/or 5-hydroxyindoleacetic acid in the CSF of the subject. The level of homovanillic acid and/or 5-hydroxyindoleacetic acid may increase at least 100% (e.g. at least 100%, at least 110%, at least 120%, at least 130%, at least 140%, at least 150%, at least 170%, at least 200%, at least 300%, or at least 400%) in the CSF of the subject compared to the level of homovanillic acid and/or 5-hydroxyindoleacetic acid prior to administration of sepiapterin, or a pharmaceutically acceptable salt thereof. Administration of sepiapterin, or a pharmaceutically acceptable salt thereof, to the subject by any of the methods described herein may also increase the concentration of serotonin and/or dopamine in the CSF of the subject. The CSF concentration of serotonin and/or dopamine of the subject may increase at least 100% % (e.g. at least 100%, at least 110%, at least 120%, at least 130%, at least 140%, at least 150%, at least 170%, at least 200%, at least 300%, or at least 400%) compared to the level of serotonin and/or dopamine prior to the administration of sepiapterin, or a pharmaceutically acceptable salt thereof.

Administration of sepiapterin, or a pharmaceutically acceptable salt thereof, to the subject by any of the methods described herein may result in an increase in nitric oxide production. The level of nitric oxide production may increase by at least 10% (e.g., at least 10%, at least 20%, at least 50%, at least 100%, at least 1000%). Without being bound by theory, the increase in nitric oxide production caused by the administration of sepiapterin, or a pharmaceutically acceptable salt thereof, may occur in tissues of interest for subjects with Parkinson's disease (e.g., the brain and nervous system, the endothelial vasculature) and may lead to decreased inflammation, improved endothelial function, and decreased oxidative stress and/or result in disease modification in Parkinson's disease progression.

Sepiapterin, or a pharmaceutically acceptable salt thereof, may or may not be administered with food. Without being bound by theory, administration of sepiapterin, or a pharmaceutically acceptable salt thereof, with food results in an increase in plasma exposure of BH4, e.g., by reducing the rate of absorption of sepiapterin. If the administered sepiapterin, or a pharmaceutically acceptable salt thereof, is absorbed quickly, e.g., by being administered on an empty stomach, sepiapterin reductase and/or dihydrofolate reductase in the cells may become saturated above Vₘₐₓ resulting in at least a portion of the administered sepiapterin leaving the cell without being reduced to 7,8-dihydrobiopterin and subsequently to BH4. This excess sepiapterin may then be excreted without ever being converted to BH4, resulting in lower levels of BH4 in the plasma compared to administration of sepiapterin with food which reduces the rate of or prolongs the absorption of sepiapterin and results in reaction rates below, at or slightly above the Vₘₐₓ for substrate saturation of sepiapterin reductase enzyme and/or dihydrofolate reductase. Administration of sepiapterin, or a pharmaceutically acceptable salt thereof, with food unexpectedly results in an increase in the maximum BH4 plasma concentration (Cₘₐₓ) and the extent of exposure as measured by the area under the concentration time curve of time zero to last concentration (AUC₀₋ₗₐₛₜ) of BH4 compared to administration without food. For example, the effective amount of sepiapterin, or a pharmaceutically acceptable salt thereof, is an amount (e.g., 2.5 mg/kg to 100 mg/kg per dose) sufficient to produce a BH4 concentration of at least 50 ng/mL (e.g., at least 60 ng/mL, at least 100 ng/mL, at least 200 ng/mL, at least 400 ng/mL, at least 600 ng/mL, at least 1000 ng/mL, or at least 2000 ng/mL or from 50 ng/mL to 100 ng/mL from 60 ng/mL to 400 ng/mL, from 200 ng/mL to 600 ng/mL, from 400 ng/mL to 1000 ng/mL, or from 600 ng/mL to 1500 ng/mL) in the plasma of the subject within 10 hours of administration with food. The effective amount may include a dose that is at least 5% (at least 10%, at least 20%, at least 50%, at least 70%, at least 90%) lower than the dose sufficient to produce a maximum BH4 plasma concentration (Cₘₐₓ) of at least 50 ng/mL (e.g., at least 60 ng/mL, at least 100 ng/mL, at least 200 ng/mL, at least 400 ng/mL, at least 600 ng/mL, at least 1000 ng/mL, or at least 2000 ng/mL or from 50 ng/mL to 100 ng/mL from 60 ng/mL to 400 ng/mL, from 200 ng/mL to 600 ng/mL, from 400 ng/mL to 1000 ng/mL, or from 600 ng/mL to 1500 ng/mL) in the plasma of the subject within 10 hours of administration of sepiapterin, or a pharmaceutically acceptable salt thereof, without food.

Administration to the subject may occur less than 30 minutes prior to consuming food or after consuming food, e.g., immediately prior to the consumption of food up to three, e.g., two, hours after consumption, such as substantially at the same time as food. The administration with food (e.g., occurring less than 30 minutes prior to consuming food or after consuming food, e.g., immediately prior to the consumption of food up to three, e.g., two, hours after consumption) may result in an increase (e.g., at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, or at least 150%) in the Cₘₐₓ of BH4 or in the extent of production and resulting plasma exposure (AUC₀₋ₗₐₛₜ) of BH4 compared to administration without food (e.g., occurring more than two hours after consuming food until 30 minutes prior to consuming further food).

Administration of sepiapterin, or a pharmaceutically acceptable salt thereof, with food may result in an increase in the maximum homovanillic acid and/or 5-hydroxyindoleacetic acid plasma concentration (Cₘₐₓ) and the extent of exposure as measured by the area under the concentration time curve of time zero to last concentration (AUC₀₋ₗₐₛₜ) of homovanillic acid and/or 5-hydroxyindoleacetic acid compared to administration without food. Administration of sepiapterin, or a pharmaceutically acceptable salt thereof, with food may increase the maximum homovanillic acid and/or 5-hydroxyindoleacetic acid plasma concentration by at least 100% (e.g. at least 100%, at least 110%, at least 120%, at least 130%, at least 140%, at least 150%, at least 170%, at least 200%, at least 300%, or at least 400%) compared to when sepiapterin is administered to the subject without food.

Administration of sepiapterin, or a pharmaceutically acceptable salt thereof, with food may result in an increase in the maximum serotonin and/or dopamine plasma concentration (Cₘₐₓ) and the extent of exposure as measured by the area under the concentration time curve of time zero to last concentration (AUC₀₋ₗₐₛₜ) of serotonin and/or dopamine compared to administration without food. Administration of sepiapterin, or a pharmaceutically acceptable salt thereof, with food may increase the maximum serotonin and/or dopamine plasma concentration by at least 100% (e.g. at least 100%, at least 110%, at least 120%, at least 130%, at least 140%, at least 150%, at least 170%, at least 200%, at least 300%, or at least 400%) compared to when sepiapterin, or a pharmaceutically acceptable salt thereof, is administered to the subject without food.

In some embodiments of any of the methods described herein, the food is a high protein food. In some embodiments of any of the methods described herein, the food is a high fat food (e.g., at least 25, 30, 40, or 50% of the calories are from fat). In some embodiments of any of the methods described herein, the food is a high protein and high fat food. In some embodiments, the food is high calorie food (e.g., the food includes at least 100 calories, e.g., at least 200 calories, at least 300 calories, at least 400 calories, at least 500 calories, e.g., 500-1500 or 800-1°±0.0 calories). In some embodiments of any of the methods described herein, the food is a meal, e.g., breakfast, lunch, or dinner. The sepiapterin, or a pharmaceutically acceptable salt thereof, may be provided in a separate composition from the consumed food (e.g., the sepiapterin, or a pharmaceutically acceptable salt thereof, is not incorporated into a food product).

The sepiapterin or salt pharmaceutically acceptable salt thereof, may be administered without food. In some embodiments of any of the foregoing methods, the sepiapterin or salt pharmaceutically acceptable salt thereof, is administered without a high protein food. In some embodiments of any of the foregoing methods, the sepiapterin, or salt pharmaceutically acceptable salt thereof, is administered without a high fat food (e.g., at least 25, 30, 40, or 50% of the calories are from fat). In some embodiments of any of the foregoing methods the sepiapterin, or pharmaceutically acceptable salt thereof, is administered without a high protein and high fat food. In some embodiments, the sepiapterin, or pharmaceutically acceptable salt thereof, is administered without a high calorie food (e.g., the food includes at least 100 calories, e.g., at least 200 calories, at least 300 calories, at least 400 calories, at least 500 calories, e.g., 500-1500 or 800-1°±0.0 calories). In some embodiments of any of the foregoing methods, the sepiapterin, or pharmaceutically acceptable salt thereof, is administered without the food being a meal, e.g., breakfast, lunch, or dinner.

Without being bound by theory, administration of sepiapterin, or salt pharmaceutically acceptable salt thereof, without food may result in an increase in plasma, CSF, and/or brain exposure of sepiapterin by increasing the rate of absorption of sepiapterin. As sepiapterin passes through cell membranes efficiently, if the administered sepiapterin is absorbed quickly, e.g., by being administered on an empty stomach, the active transporters of sepiapterin and/or sepiapterin reductase enzymes in cells may be saturated resulting in at least a portion of the administered sepiapterin not entering the cells and/or leaving the cell without being reduced to 7,8-dihydrobiopterin. This excess sepiapterin in the plasma may then cross the blood brain barrier (BBB) and enter into brain cells prior to being converted to BH4, resulting in higher levels of BH4 in the brain (and/or CSF) compared to administration with food, which reduces the rate of absorption of sepiapterin and may not result in saturation of the sepiapterin transporters and intracellular sepiapterin reductase enzymes. Thus, administration of sepiapterin, or salt pharmaceutically acceptable salt thereof, without food unexpectedly results in an increase in the maximum plasma, CSF, and/or brain concentration (Cₘₐₓ) and/or the extent of absorption (AUC₀₋ₗₐₛₜ) of sepiapterin compared to administration with food. The increased levels of sepiapterin in the plasma, CSF, and/or brain may be beneficial in the treatment of certain BH4 disorders, e.g., CNS disorders related to BH4.

Administration of sepiapterin, or a pharmaceutically acceptable salt thereof, to the subject by any of the methods described herein may result in improvements in quality of life, by improvements in mood, sleep, cognition, behavior, and/or symptoms. Improvements may result after the subject is administered sepiapterin, or a pharmaceutically acceptable salt thereof, over a period of time e.g., 1 week, 2 weeks, 1 month, 2 months, 6 months, or 1 year. The treatment may produce an improvement in mood, resulting in an improvement in the Hamilton Depression Rating Scale in the subject, such that the score measured for a subject after being administered sepiapterin, or a pharmaceutically acceptable salt thereof, is lower (e.g., at least 10% lower, at least 20% lower, at least 30% lower, at least 40% lower, at least 50% lower, at least 60% lower, at least 70% lower, at least 80% lower, or at least 90% lower) on an integer scale of 0 to 52, wherein 0 is considered no depression and 52 is considered severe depression, compared to before being administered sepiapterin, or a pharmaceutically acceptable salt thereof. The treatment may produce an improvement in sleep quality and may result in an improvement in the Parkinson's Disease Sleep Scale in the subject, such that the score measured for a subject after being administered sepiapterin is higher (e.g., at least 10% higher, at least 20% higher, at least 30% higher, at least 40% higher, at least 50% higher, at least 60% higher, at least 70% higher, at least 80% higher, at least 90% higher, or at least 100% higher) on an integer scale of 0 to 10 for 15 points of assessment, wherein 0 is awful sleep and 10 is excellent sleep, compared to before being administered sepiapterin, or a pharmaceutically acceptable salt thereof. The treatment may produce an improvement in the symptoms experienced by the subject, such that Total Daily OFF Time is decreased, e.g., the total number of hours per day wherein the treatment administered to the subject does not adequately manage the symptoms associated with Parkinson's, after a subject is administered sepiapterin compared to before a subject is administered sepiapterin, or a pharmaceutically acceptable salt thereof. The treatment may increase in neurocognitive function of the subject (e.g., an increase in executive function, a decrease in anxiety, a decrease in attention-deficit/hyperactivity disorder symptoms, and/or a decrease in instances of brain fog). Changes in executive function may be determined by an assessment, such as Behavioral Assessment of Dysexecutive Syndrome (BADS), Behavior Rating Inventory of Executive Function (BRIEF), Barkley Deficits in Executive Functioning Scales (BDEFS), Behavioral Dyscontrol Scale (BDS), Comprehensive Executive Function Inventory (CEFI), CogScreen, Continuous Performance Task (CPT), Controlled Oral Word Association Test (COWAT), d2 Test of Attention, Delis-Kaplan Executive Function System (D-KEFS), Digit Vigilance Test, Figural Fluency Test, Halstead Category Test, Hayling and Brixton tests, Iowa gambling task, Kaplan Baycrest Neurocognitive Assessment (KBNA), Kaufman Short Neuropsychological Assessment, Paced Auditory Serial Addition Test (PASAT), Rey-Osterrieth Complex Figure, Ruff Figural Fluency Test, Stroop task, Tasks of Executive Control, Test of Variables of Attention (T.O.V.A.), Tower of London Test, Trail-Making Test (TMT) or Trails A & B, Wisconsin Card Sorting Test (WCST), Symbol Digit Modalities Test, or a Cambridge Neuropsychological Test Automated Battery (CANTAB) assessment, e.g., by measuring reaction time, spatial span, spatial working memory, rapid digital information processing, sustained attention, and/or stop signal task. The treatment may produce an improvement in attention and/or mood, e.g., as measured by the ADHD-RS IV scale (or the Inattention assessment thereof) and/or the Profile Mood States (POMS) scale.

### EQUIVALENTS AND SCOPE

Those skilled in the art will recognize or be able to ascertain using no more than routine experimentation, many equivalents to the specific embodiments in accordance with the invention described herein. The scope of the present invention is not intended to be limited to the above Description, but rather is as set forth in the appended claims.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any compound; any method of production; any method of use; etc.) can be excluded from any one or more claims, for any reason, whether or not related to the existence of prior art.

### EXAMPLES

### Example 1: Evaluation of the Food Effect on the Administration of Sepiapterin

Method: Subjects received two oral doses of sepiapterin (10 mg/kg) separated by one week in a fasted and fed state. Subjects were fed a standard high-fat (approximately 50 percent of total caloric content of the meal) and high-calorie (approximately 800 to 1°±0.0 calories) meal starting 30 minutes prior to receiving their second oral dose of sepiapterin on Day 8.

Sampling for PK analysis occurred predose on Days 1 and 8 (within 30 minutes before dosing) and 0.5 hr, 1 hr, 2 hr, 4 hr, 8 hr, 12 hr, and 24 hr after the doses on Days 1 and 8. The blood concentrations of sepiapterin and BH4 were analyzed by MNG Labs.

The cerebrospinal fluid (CSF) of selected subjects was collected via lumbar puncture on Day1 (before dose) and on Day 7 (i.e., following daily dosing for 7 days) approximately 30 minutes from time of the maximum observed plasma BH4 concentration (Tmax) as determined from the blood analysis.

The cerebral spinal fluid (CSF) was analyzed. Descriptive statistics are provided to characterize any changes in neurotransmitter metabolism between the Day 1, and Day 7 sample results.

Results: As shown in Tables 24 and 25 below, the Cₘₐₓ of BH4 in the plasma was much higher in subjects when fed prior to administration compared to subjects who had fasted prior to administration. Further, there was a decrease in plasma sepiapterin concentration but an increase in BH4 concentration, when sepiapterin was administered in the fed vs. fasting state. Geometric mean ratios (fasted/fed, 90% CI) for plasma sepiapterin were 1.29 (0.84 to 2.00) for AUCₗₐₛₜ and 1.57 (1.21 to 2.0) for Cₘₐₓ. Corresponding ratios (90% CI) for plasma BH4 were 0.58 (0.47 to 0.71) for AUC_{0-inf}, and 0.55 (0.45 to 0.68) for Cₘₐₓ. Overall exposure to BH4 as measured by AUC_{0-inf} and AUCₗₐₛₜ increased by 1.7-fold when sepiapterin was administered in the fed state compared with the fasted state.

TABLE 24
Summary of BH4 Concentration in Plasma of Fasted Subjects

| | Time (hours) | | | | | | | |
|---|---|---|---|---|---|---|---|---|
| | 0 | 0.5 | 1 | 2 | 4 | 8 | 12 | 24 |
| Mean (nM) | 7.62 | 25.65 | 154.63 | 413.48 | 624.34 | 248.09 | 85.87 | 18.33 |
| SD (nM) | 1.26 | 14.20 | 42.65 | 104.49 | 210.24 | 118.04 | 39.26 | 8.65 |
| Median (nM) | 7.75 | 22.85 | 147.30 | 370.40 | 635.05 | 232.55 | 75.30 | 15.35 |
| Minimum (nM) | 5.00 | 13.80 | 98.50 | 271.40 | 379.00 | 90.20 | 38.90 | 8.50 |
| Maximum (nM) | 9.00 | 66.50 | 223.10 | 617.90 | 1127.90 | 457.30 | 162.00 | 37.40 |

TABLE 25
Summary of BH4 Concentration in Plasma of Fed Subjects

| | Time (hours) | | | | | | | |
|---|---|---|---|---|---|---|---|---|
| | 0 | 0.5 | 1 | 2 | 4 | 8 | 12 | 24 |
| Mean (nM) | 8.43 | 33.32 | 165.79 | 586.90 | 1098.85 | 520.33 | 161.39 | 19.27 |
| SD (nM) | 1.45 | 21.70 | 82.02 | 249.26 | 289.74 | 192.50 | 58.43 | 8.20 |
| Median (nM) | 8.50 | 30.95 | 160.00 | 554.00 | 1010.60 | 485.00 | 142.30 | 16.15 |
| Minimum (nM) | 6.00 | 8.90 | 42.30 | 298.90 | 750.80 | 252.70 | 90.40 | 12.10 |
| Maximum (nM) | 11.00 | 76.40 | 369.00 | 1199.90 | 1566.70 | 926.30 | 261.10 | 40.10 |

Further, as shown in Tables 26 and 27 below, the Cₘₐₓ of sepiapterin in the plasma was much lower in subjects when fed prior to administration compared to subjects who fasted prior to administration.

TABLE 26
Summary of Sepiapterin Concentration in Plasma of Fasted Subjects

| | Time (hours) | | | | | | | |
|---|---|---|---|---|---|---|---|---|
| | 0 | 0.5 | 1 | 2 | 4 | 8 | 12 | 24 |
| Mean (nM) | 0 | 2.85 | 5.43 | 4.63 | 1.88 | 0 | 0 | 0 |
| SD (nM) | 0 | 2.67 | 2.22 | 1.36 | 1.48 | 0 | 0 | 0 |
| Median (nM) | 0 | 2.32 | 4.82 | 4.71 | 2.37 | 0 | 0 | 0 |
| Minimum (nM) | 0 | 0 | 3.15 | 2.52 | 0 | 0 | 0 | 0 |
| Maximum (nM) | 0 | 7.23 | 10.19 | 6.65 | 3.93 | 0 | 0 | 0 |

TABLE 27
Summary of Sepiapterin Concentration in Plasma of Fed Subjects

| | Time (hours) | | | | | | | |
|---|---|---|---|---|---|---|---|---|
| | 0 | 0.5 | 1 | 2 | 4 | 8 | 12 | 24 |
| Mean (nM) | 0 | 0.63 | 2.38 | 2.96 | 2.96 | 0.18 | 0 | 0 |
| SD (nM) | 0 | 1.17 | 1.66 | 1.88 | 1.53 | 0.63 | 0 | 0 |
| Median (nM) | 0 | 0 | 2.42 | 2.91 | 2.50 | 0 | 0 | 0 |
| Minimum (nM) | 0 | 0 | 0 | 0 | 0 | 0 | 0 | 0 |
| Maximum (nM) | 0 | 3.22 | 4.57 | 5.92 | 5.56 | 2.19 | 0 | 0 |

Further as shown in Table 28, the AUC₀₋₂₄ and AUC_{0-inf} was much higher in fasted subject administered sepiapterin than subjects administered BH4. Six subjects received sepiapterin in each group and three subjects received BH4 in each group where this treatment was administered.

TABLE 28
Summary of pharmacokinetic parameters for BH4 after administration
of single dose of sepiapterin or BH₄ in fasted subjects.

| | | Dose of sepiapterin or BH4 (mg/kg) | | | | |
|---|---|---|---|---|---|---|
| Parameter | Treatment | 2.5 | 7.5 | 20 | 40 | 80 |
| Cₘₐₓ | sepiapterin | 57 | 101 | 138 | 173 | 321 |
| (ng/mL) | BH4 | 16 | 46 | 84 | NT | NT |
| Tₘₐₓ (h) | sepiapterin | 3.7 | 3.7 | 3.7 | 4.0 | 3.7 |
| | BH4 | 4.0 | 4.1 | 2.3 | NT | NT |
| AUC₀₋₂₄ | sepiapterin | 397 | 762 | 943 | 1204 | 1980 |
| (ng.h/mL) | BH4 | 162 | 355 | 612 | NT | NT |
| AUC_{0-inf} | sepiapterin | 430 | 784 | 1064 | 1170 | 2080 |
| (ng.h/mL) | BH4 | 248 | 363 | 644 | NT | NT |

Values shown are means of plasma concentrations of BH4 following administration at the doses shown.

### Example 2. Comparison of Adverse Events in Fed and Fasted Subjects

Method: Twelve subjects were given a single dose (10 mg/kg) of sepiapterin under fasting conditions and then 7 days later under fed conditions. Standard definitions of adverse events (AE) were used. All-cause AE were those that occurred at any time; treatment-emergent adverse events (TEAE) were those that occurred at or after the time of administration of study treatment. TEAEs related to study drug were based on the opinion of the investigator. Serious AE were defined as life threatening or caused death, hospitalization or prolongation of existing hospitalization, or were a persistent or significant disability/incapacity or a substantial disruption of the ability to conduct normal life functions, or a congenital anomaly/birth defect.

Results: As shown in Table 29, surprisingly, there was a decreased incidence of adverse events when sepiapterin was administered to fed subjects compared to fasted subjects.

TABLE 29
Comparison of adverse events in fasted and fed conditions

| | Sepiapterin 10 mg/kg | |
|---|---|---|
| | Fasted | Fed |
| ≥1 AE | 5 | 1 |
| TEAE | 4 | 1 |
| TEAE related to study drug | 1 | 0 |

### Example 3. Determination of CSF Levels of Neurotransmitters after Administration of Sepiapterin

Method: Analyses were conducted in CSF samples of subjects administered 60 mg/kg sepiapterin or placebo. The following analytes were measured: sepiapterin, BH4, BH2, homovanillic acid (HVA), and 5-hydroxyindoleacetic acid (HIAA). Descriptive (n, mean, SD, median, minimum, maximum) statistics for these analytes at Day 1 and Day 7+/−30 minutes from Tmax and change from baseline at Day 7 were determined for each analyte.

Results: Sepiapterin was not detected in the CSF on Day 1 or Day 7+/−30 minutes from BH4 Tmax. The CSF concentration of BH4 increased by 4.102 ng/mL in sepiapterin-treated subjects while it did not change in subjects receiving placebo (change from baseline of −0.010 ng/mL). Similarly, the CSF concentration of BH2 increased by 1.368 ng/mL in sepiapterin-treated subjects while it did not change in subjects receiving placebo (change from baseline of −0.020 ng/mL).

Changes in neurotransmitter concentrations relative to Day 1 also differ between sepiapterin-treated subjects and subjects receiving placebo. The concentration of HVA increased by 1.378 ng/mL in sepiapterin-treated subjects compared with a decrease of 0.630 ng/mL in subjects receiving placebo. The concentration of 5-HIAA decreased in both arms but to a lesser extent in sepiapterin-treated subjects than in subjects receiving placebo (−1.142 ng/mL versus −2.440 ng/mL).

Of note, Day 1 pre-dose HVA (10.02 ng/ml) and 5-HIAA (3.69 ng/mL) concentrations for one subject were approximately a third of the concentrations observed in the other subjects (HVA: 22.45 ng/mL to 44.72 ng/mL; 5-HIAA: 9.49 ng/mL to 19.70 ng/mL). On Day 7, however, HVA (29.93 ng/mL) and 5-HIAA (9.53 ng/mL) concentrations for this subject were close to the range observed in the other sepiapterin administered subjects (HVA: 30.64 ng/mL to 43.19 ng/mL; 5-HIAA: 10.10 ng/mL to 21.59 ng/mL). HVA and 5-HIAA concentrations for the subject on Day 7 were both higher than the mean concentrations for Day 7 placebo-treated subjects. The data is summarized in Table 30 below.

TABLE 30
Analyte concentrations in the CSF of subjects.

| Analyte | Day | T reatment | Mean Concentration (ng/mL) |
|---|---|---|---|
| 5-HIAA | 1 | sepiapterin | 14.6 |
| | | placebo | 11.9 |
| | 7 | sepiapterin | 13.4 |
| | | placebo | 9.5 |
| BH2 | 1 | sepiapterin | 0.5 |
| | | placebo | 0.5 |
| | 7 | sepiapterin | 1.9 |
| | | placebo | 0.5 |
| BH4 | 1 | sepiapterin | 4.4 |
| | | placebo | 3.2 |
| | 7 | sepiapterin | 8.5 |
| | | placebo | 3.2 |
| HVA | 1 | sepiapterin | 32.3 |
| | | placebo | 25.1 |
| | 7 | sepiapterin | 33.7 |
| | | placebo | 24.5 |
| Sepiapterin | 1 | sepiapterin | 0 |
| | | placebo | 0 |
| | 7 | sepiapterin | 0 |
| | | placebo | 0 |

### Example 4. Pharmacokinetic Analysis of Administration of Multiple Doses of Sepiapterin

Methods: Three cohorts of eight fed subjects each were randomized to receive once-daily sepiapterin or placebo for 7 days in a 6:2 ratio. A sentinel dose strategy was also used for administration of the highest sepiapterin dose.

Results: Plasma-time concentrations of sepiapterin and BH4 were similar after 1 and 7 days of treatment with sepiapterin, with no drug accumulation. The pharmacokinetic data is shown in Table 31 below.

TABLE 31
Summary of pharmacokinetic data for multiple
administrations of sepiapterin

| | Dose of sepiapterin (mg/kg) | | | | |
|---|---|---|---|---|---|
| Parameter | Compound | Day | 5 | 20 | 60 |
| Cₘₐₓ | sepiapterin | 1 | 0.6 | 1.2 | 2.7 |
| (ng/mL) | | 7 | 0.6 | 1.3 | 2.8 |
| | BH4 | 1 | 147 | 496 | 597 |
| | | 7 | 152 | 516 | 678 |
| Tmax (h) | sepiapterin | 1 | 3.0 | 2.6 | 3.3 |
| | | 7 | 3.0 | 2.7 | 3.0 |
| | BH4 | 1 | 4.0 | 4.0 | 4.6 |
| | | 7 | 4.0 | 4.0 | 4.0 |
| AUC₀₋₂₄ | sepiapterin | 1 | NC | NC | 23 |
| (ng.h/mL) | | 7 | NC | NC | 23 |
| | BH4 | 1 | 994 | 3031 | 4560 |
| | | 7 | 1070 | 3718 | 4864 |
| AUC_{0-inf} | sepiapterin | 1 | 1.0 | 3.9 | 21.2 |
| (ng.h/mL) | | 7 | 0.7 | 4.3 | 16.0 |
| | BH4 | 1 | 1014 | 3085 | 4668 |
| | | 7 | NC | NC | NC |

NC = Not calculated

### Example 5: Treatment of Subjects Diagnosed with Parkinson's Disease

According to the methods described herein, a physician or one of skill in the art can treat a subject, such as a human subject, so as to reduce or alleviate symptoms of Parkinson's disease. To this end, a human subject can be administered sepiapterin for the subject to self-administer once daily (or in some embodiments twice daily), for a period of 14 days. The sepiapterin will be administered orally, e.g., as a suspension of sepiapterin in Medisca Oral Mix or a tablet, capsule, or caplet, and will be administered with food, such as a morning and/or evening meal. The dosage of sepiapterin will be adjusted according to the body weight of the subject such that the daily dosage is 60 mg/kg/day. The subject's weight will be determined upon screening to calculate the appropriate dosage for the subject. Assessments of the treatment will be made on the first, second, and fourteenth day of treatment to determine the effectiveness of treatment.

The effectiveness of treatment will be evaluated according to both pharmacodynamic (PD) parameters, such as blood concentrations of serotonin and dopamine and cerebral spinal fluid concentrations of BH4, homovanillic acid, and 5-hydroxyindoleactetic acid, as well as qualitative subject assessment of symptoms, such as the Hamilton Depression Rating Scale, the MDS-UPDRS, the Parkinson's Sleep Scale, and Total OFF time. The concentration of BH4 in cerebral spinal fluid will be assessed by way of lumbar puncture immediately prior to administration and thirty minutes, one hour, two hours, four hours, and eight hours prior to administration on the second and fourteenth day of being administered the sepiapterin. Additionally, blood will be collected immediately before or concurrently with the lumbar punctures on the first day of administration and prior to administration on the fourteenth day.

Pharmacodynamic endpoints will be changes from baseline of homovanillic acid, 5-hydroxyindoleactetic acid, BH4, BH2, sepiapterin, and neopterin in the cerebral spinal fluid, where baseline measurements were determined prior to treatment. Additional, pharmacodynamic endpoints will be assessed as changes from baseline in serotonin and dopamine in whole blood plasma. The efficacy of the treatment is measured as changes from the baseline in the MDS-UPDRS and in the Total Daily OFF time on the fourteenth day of administration. Additional reduction in symptoms may result in changes from baseline in the Hamilton Depression Rating Scale and the Parkinson's Disease Sleep Scale.

### Example 6: Treatment of Subjects Diagnosed with Parkinson's Disease

According to the methods described herein, a physician or one of skill in the art can treat a subject, such as a human subject, so as to reduce or alleviate symptoms of Parkinson's disease. To this end, a human subject can be administered sepiapterin for the subject to self-administer once daily (or in some embodiments twice daily), for a period of 14 days. The sepiapterin will be administered orally, e.g., as a suspension of sepiapterin in Medisca Oral Mix or a tablet, capsule, or caplet, and will be administered without food, such as more than 30 minutes before or 2 hours after a morning and/or evening meal. The dosage of sepiapterin will be adjusted according to the body weight of the subject such that the daily dosage is 60 mg/kg/day. The subject's weight will be determined upon screening to calculate the appropriate dosage for the subject. Assessments of the treatment will be made on the first, second, and fourteenth day of treatment to determine the effectiveness of treatment.

The effectiveness of treatment will be evaluated according to both pharmacodynamic (PD) parameters, such as blood concentrations of serotonin and dopamine and cerebral spinal fluid concentrations of BH4, homovanillic acid, and 5-hydroxyindoleactetic acid, as well as qualitative subject assessment of symptoms, such as the Hamilton Depression Rating Scale, the MDS-UPDRS, the Parkinson's Sleep Scale, and Total OFF time. The concentration of BH4 in cerebral spinal fluid will be assessed by way of lumbar puncture immediately prior to administration and thirty minutes, one hour, two hours, four hours, and eight hours prior to administration on the second and fourteenth day of being administered the sepiapterin. Additionally, blood will be collected immediately before or concurrently with the lumbar punctures on the first day of administration and prior to administration on the fourteenth day.

Pharmacodynamic endpoints will be changes from baseline of homovanillic acid, 5-hydroxyindoleactetic acid, BH4, BH2, sepiapterin, and neopterin in the cerebral spinal fluid, where baseline measurements were determined prior to treatment. Additional, pharmacodynamic endpoints will be assessed as changes from baseline in serotonin and dopamine in whole blood plasma. The efficacy of the treatment is measured as changes from the baseline in the MDS-UPDRS and in the Total Daily OFF time on the fourteenth day of administration. Additional reduction in symptoms may result in changes from baseline in the Hamilton Depression Rating Scale and the Parkinson's Disease Sleep Scale.

What is claimed is:

1. A method of treating Parkinson's disease in a subject in need thereof, the method comprising administering to the subject an effective amount of sepiapterin, or a pharmaceutically acceptable salt thereof.

2. The method of claim 1, comprising administering to the subject an effective amount of sepiapterin, or a pharmaceutically acceptable salt thereof, with food.

3. The method of claim 1, comprising administering to the subject an effective amount of sepiapterin, or a pharmaceutically acceptable salt thereof, without food.

4. A method of treating Parkinson's disease in a subject in need thereof, the method comprising administering to the subject an effective amount of sepiapterin, or a pharmaceutically acceptable salt thereof, once per day.

5. A method of treating Parkinson's disease in a subject in need thereof, the method comprising administering to the subject an effective amount of sepiapterin, or a pharmaceutically acceptable salt thereof, more than once per day.

6. A method of treating Parkinson's disease in a subject in need thereof, the method comprising administering about 50 mg/kg to about 100 mg/kg of sepiapterin per day, or a pharmaceutically acceptable salt thereof to the subject.

7. A method of treating Parkinson's disease in a subject in need thereof, the method comprising administering to the subject an effective amount of sepiapterin, or pharmaceutically acceptable salt thereof, in with combination with at least one other agent used to treat Parkinson's disease.

8. The method of any one of claims 1 or 5-7, wherein the method comprises administering to the subject an effective amount of sepiapterin, or a pharmaceutically acceptable salt thereof, with food twice per day.

9. The method of any one of claims 1 or 5-7, wherein the method comprises administering to the subject an effective amount of sepiapterin, or a pharmaceutically acceptable salt thereof, without food twice per day.

10. The method of any one of claims 1 to 9, wherein the effective amount is an amount sufficient to produce a BH4 concentration of at least 50 ng/mL in the plasma of the subject within 10 hours of administration.

11. The method of claim 2, wherein the effective amount comprises a dose that is at least 20% lower than the dose sufficient to produce a maximum BH4 plasma concentration (Cₘₐₓ) of at least 50 ng/mL in the plasma of the subject within 10 hours of administration of sepiapterin, or a pharmaceutically acceptable salt thereof, without food.

12. The method of any one of claims 1 to 11, wherein the effective amount is 2.5 mg/kg to 100 mg/kg per dose.

13. The method of any one of claims 1 to 2, 4 to 8, or 10 to 12, wherein the administration to the subject occurs less than 30 minutes prior to consuming food.

14. The method of any one of claims 1 to 2, 4 to 8, or 10 to 12, wherein the administration to the subject is substantially at the same time as food.

15. The method of any one of claims 1 to 2, 4 to 8, or 10 to 12, wherein the administration is immediately after the consumption of food up to 2 hours after the consumption.

16. The method of any one of claims 1, 3 to 7, 9 to 10, or 12, wherein the administration to the subject occurs more than 30 minutes prior to consuming food.

17. The method of any one of claims 1, 3 to 7, 9 to 10, 12, or 16, wherein the administration is more than 2 hours after the consumption of food.

18. The method of any one of claims 1 to 17, wherein the effective amount results in an increase in the level of homovanillic acid and/or 5-hydroxyindoleacetic acid in the CSF of the subject.

19. The method of any one of any one of claims 1 to 18, wherein the level of homovanillic acid and/or 5-hydroxyindoleacetic acid in the subject is increased at least 100% compared to the level prior to administration.

20. The method of any one of claims 1 to 19, wherein the effective amount results in an increase in the level of serotonin and/or dopamine in the CSF of the subject.

21. The method of any one of claims 1 to 20, wherein the level of serotonin and/or dopamine in the subject is increased at least 100% compared to the level prior to administration.

22. The method of any one of claims 1 to 21, wherein the sepiapterin or a pharmaceutically acceptable salt thereof is formulated as an oral powder for suspension.

23. The method of any one of claims 1 to 22, wherein the sepiapterin or a pharmaceutically acceptable salt thereof is administered as a suspension in Medisca Oral Mix.

24. The method of any one of claims 1 to 23, wherein the method further comprises administering an effective amount of levodopa to the subject.

25. The method of any one of claims 1 to 24, wherein the method further comprises administering an effective amount of carbidopa to the subject.

26. The method of any one of claims 1 to 25, wherein the method further comprises administering an effective amount of an AADC inhibitor, an AADC gene therapy, a COMT inhibitor, a dopamine agonist, a MAO-B inhibitor, a DAT inhibitor, a NMDA antagonist, an anticholinergic agent, and/or an acetylcholinesterase inhibitor.

27. The method of any one of claims 1 to 26, wherein the effective amount of sepiapterin comprises 60 mg/kg per day.

28. The method of claim 27, wherein the 60 mg/kg per day is administered in two equal doses of 30 mg/kg.

29. The method of claim 27, wherein the 60 mg/kg per day is administered in two unequal doses of 40 mg/kg and 20 mg/kg.

30. The method of claim 28, wherein the two equal doses are administered with a morning meal and with an evening meal.

31. The method of claim 29, wherein the two unequal doses are administered with a morning meal and with an evening meal.

32. The method of any one of claims 1 to 31, wherein the administration results in an improvement in the MDS-UPDRS of the subject.

33. The method of any one of claims 1 to 32, wherein the administration results in a decrease in the Total Daily OFF Time in the subject.

34. The method of any one of claims 1 to 33, wherein the administration results in an improvement in the Hamilton Depression Rating Scale in the subject.

35. The method of any one of claims 1 to 34, wherein the results in an improvement in the Parkinson's Disease Sleep Scale in the subject.
